(19) United States
(12) Patent Application Publication
(10) Pub. No.: US 2010/0029909 A1
(43) Pub. Date: Feb. 4, 2010

(54) COMPOSITIONS AND METHODS COMPRISING MAGNETIC RESONANCE CONTRAST AGENTS

(75) Inventors: Preeti A. Sukerkar, Glenview, IL (US); Jiyoun Lee, Palo Alto, CA (US); Teresa K. Woodruff, Chicago, IL (US); Thomas J. Meade, Wilmette, IL (US)

Correspondence Address: Casimir Jones, S.C., 2275 DEMING WAY, SUITE 310, MIDDLETON, WI, 53562, US

(73) Assignee: NORTHWESTERN UNIVERSITY, Evanston, IL (US)

(21) Appl. No.: 12/471,731

(22) Filed: May 26, 2009

Related U.S. Application Data
(60) Provisional application No. 61/055,629, filed on May 23, 2008.

(51) Int. Cl. C07K 14/575; C07K 14/435; G01R 33/48

(52) U.S. Cl. 530/399; 530/400; 324/309

(57) ABSTRACT

The present invention relates to compositions and methods for imaging with magnetic resonance contrast agents. In particular, the present invention provides targeted contrast agents for selective imaging.

### CROSS-REFERENCE TO RELATED APPLICATIONS

The present application claims priority to U.S. Provisional Application Ser. No. 61/055,629 filed May 23, 2008, which is herein incorporated by reference.

### STATEMENT REGARDING FEDERALLY SPONSORED RESEARCH OR DEVELOPMENT

This invention was made with government support under National Institutes of Health Grant Nos. 1 R01 EB005866-01, 5 U54 CA090810, and R01 HD044464. The government has certain rights in the invention.

### FIELD OF THE INVENTION

The present invention relates to compositions and methods for imaging with magnetic resonance contrast agents. In particular, the present invention provides targeted contrast agents for selective imaging.

### BACKGROUND

An important tool in clinical diagnosis of disease is the use of the magnetic resonance imaging (MRI) contrast agents. In MRI, images are acquired by employing radio frequency pulses to excite nuclear spins of a specimen. The observed signal is from the protons of water molecules in the specimen. MRI can capture three-dimensional images without the need for invasive procedures. By imposing one or more orthogonal magnetic field gradients onto a target, an MR image can be obtained using radio frequency pulses to excite nuclear spins as in NMR. Images are based upon the NMR signal from the protons of water molecules, where the signal intensity in a given volume element is a function of the water concentration and relaxation times (T₁ and T₂).

MRI has several advantages over other imaging modalities. MRI can image in three dimensions with high spatial and temporal resolution. Unlike while light microscopy and fluorescent microscopy, MRI is not limited by the distance of scattered light onto the cells of interest or dye intensity. It avoids the harmful ionizing radiation of X-ray and CT. Finally, while positron emission tomography (PET) has higher sensitivity, the resolution is much lower than for MRI. MRI can visualize opaque organisms in three dimensions and can follow organisms over time, making it an ideal biological imaging tool.

Sensitivity and intrinsic contrast can be enhanced by using paramagnetic contrast agents, such as the commonly used paramagnetic Gd(III) ion, that decreases the local T₁ relaxation of nearby water protons (Caravan et al. Chem Rev 1999, 99, 2293-352., herein incorporated by reference in its entirety). Images derived from changes in T₁ regions that are associated with a Gd(III) ion have a higher signal intensity (Aime et al. Curr Pharm Biotechnol 2004, 5, 509-18., herein incorporated by reference in its entirety). The ability of a contrast agent to decrease T₁ and therefore increase signal intensity at a given concentration is relaxivity (mM⁻¹ s⁻¹). High relaxivity agents result in area of increased signal.

Free Gd(III) ions are toxic to biological systems, and a suitable ligand or chelate must bind the lanthanide to form a nontoxic complex. For many years, Gd(III) contrast agents have been used in a chelated form to eliminate toxicity in humans. Several factors influence the stability of chelate complexes including enthalpy and entropy effects (e.g., number, charge and basicity of coordinating groups, ligand field, and conformational effects). Recently, there has been concern associated with the use of Gd(III) agents due to an apparent link to a disabling condition called NSF (Kurtkoti & Hiremagalur. Nephrology 2008, 13, 235-41., Kay. Ann Rheum Dis 2008, 67 Suppl 3, iii66-9., herein incorporated by reference in their entirety). Many patients with this condition experience a thickening of the skin that inhibits joint movement. Clinical data on known cases of NSF have revealed that the condition is only present in patients with renal failure (low pH) and only when certain classes of contrast agent are used. Three clinically approved contrast agents have been associated with the onset of NSF: OMNISCAN, MAGNEVIST and OPTIMARK. These are all linear Gd(III) chelates based on the structure of DTPA.

Macrocyclic chelates have higher thermodynamic stability constants and have not been associated with NSF. The reduced thermodynamic stability constants (and the presence of an amide) in linear chelates is thought to be the cause of NSF when Gd(III) is released from the chelate and displaced by other naturally occurring metals. The medical community has studied the risks of using gadolinium in patients with renal failure and has published guidelines to minimize the risk (Shellock & Spinazzi, AJR Am J Roentgenol 2008, 191, 1129-39., herein incorporated by reference in its entirety).

Gd(III) ions are toxic to living tissues, presumably due to binding to calcium channels and therefore, it must be chelated to reduce the bioavailability. These chelates are synthetically versatile and provide the means to attach targeting moieties (Allen & Meade. Met Ions Biol Syst 2004, 42, 1-38., herein incorporated by reference in its entirety).

Mammary epithelial cells express the progesterone receptor (PR) and estrogen receptors (ER) (Ismail et al. Steroids 2003, 68, 779-87., herein incorporated by reference in its entirety). The PR is present in two distinct isoforms both derived from the same gene, PRA and PRB. Each subtype is critical to mammary gland lobuloalveolar development and epithelial differentiation (Lanari & Molinolo Breast Cancer Res 2002, 4, 240-3., herein incorporated by reference in its entirety). The receptor consists of several regions that serve as functional units such as the DNA binding domain (DBD), the ligand binding domain (LBD), and transcriptional activation domains (AFs). The expression of these receptors is a critical parameter typically examined using immunohistochemistry in biopsies of human breast cancers (Jacobsen et al. J Mammary Gland Biol Neoplasia 2003, 8, 257-68., herein incorporated by reference in its entirety).

The presence of both receptors correlates significantly with the survival rate of breast cancer patients (Hopp et al. Clin Cancer Res 2004, 10, 2751-60., herein incorporated by reference in its entirety). The PR is an estrogen-regulated gene that becomes activated and expressed in the presence of estradiol and ER. Therefore, it is not surprising that treatment with tamoxifen (an anti-estrogenic therapy) reduces PR. Decreased PR correlates with tamoxifen resistance, although the mechanism of resistance is still being debated (Arpino et al. J Natl Cancer Inst 2005, 97, 1254-61., herein incorporated by reference in its entirety). Tumors that are ER+/PR− are considered more metastatic and aggressive than PR+ tumors and correlate with a lower survival rate (Cui et al. J Clin Oncol 2005, 23, 7721-35., herein incorporated by reference in its entirety).

An important prognostic marker is the presence of ER+/PR− tumors because these cancers respond much better to aromatase inhibitors than ER+/PR+ tumors that can be effectively treated with tamoxifen (Fuqua et al. J Clin Oncol 2005, 23, 931-2; author reply 932-3., Osborne et al. Breast 2005, 14, 458-65., herein incorporated by reference in their entireties). In addition, expression of PR may also reflect activation of the growth factor pathway Her2/neu. Since monoclonal antibodies directed against the Her2/neu receptor are being developed for breast cancer, knowing the PR status might help determine if a patient will respond to these treatments (Montemurro & Aglietta, Clin Breast Cancer 2005, 6, 77-80., herein incorporated by reference in its entirety). Therefore, non-invasively delineating whether or not a mammary cancer expresses the PR may be crucial to determining the best chemotherapeutic agent for the patient and ultimately improve survival.

The progression of endometrial cancer resembles that of breast cancer in regards to the expression of progesterone receptors. For example, the loss of both PRA and PRB is associated with a poor prognosis and inversely correlated with disease free survival (Boruban et al. Eur J Cancer Prev 2008, 17, 133-8., Uharcek. Obstet Gynaecol Res 2008, 34, 776-83., herein incorporated by reference in their entireties). When each isoforms of the receptor is analyzed separately, the correlation to disease progression is less clear (Arnett-Mansfield et al. Cancer Res 2001, 61, 4576-82., herein incorporated by reference in its entirety); however, the contrast agent would bind to all available progesterone receptors and therefore the overall loss of PR is more critical for imaging purposes. In cell lines, a reduction in the level of PRs is associated with increases in genes that regulate invasion (Miyamoto et al. J Steroid Biochem Mol Biol 2004, 92, 111-8., Saito et al. Cancer Sci 2006, 97, 1308-14., herein incorporated by reference in their entireties). Further, in tissue samples PR is inversely correlated to Ki67, a marker of cellular proliferation. Progesterone is also a therapeutic agent for endometrial cancers with many patients receiving progesterone to slow the growth of their cancer (Ito et al. Endocr J 2007, 54, 667-79., herein incorporated by reference in its entirety).

Recent reports have addressed the use of MR imaging for analyzing breast tumors (Lehman et al. N Engl J Med 2007, 356, 1295-303., Tozaki. Breast Cancer 2008, 15, 205-11., Lee et al. Radiology 2008, 246, 763-71., herein incorporated by reference in their entireties). Although the majority of physicians conclude that traditional mammography is the best for routine screening of the general population, MR imaging is increasingly used in tumor imaging. For patients with familial risk of breast cancer lesions tend to form quickly and have varying appearance using mammography. When a patient has a positive mammography and biopsy, MR imaging is a second line technique to discover other lesions and identify lesions in the contralateral breast. MR can be helpful for guiding biopsies so that the needle is inserted directly into the cancerous area for accurate results.

MR imaging is valuable for determining if a patient is responding to therapy. Response to therapy is one of the critical areas that a targeted steroid-based contrast agent can be used because many drugs for breast cancers down regulate estrogen inducible genes, such as the progesterone receptor. For uterine cancers in particular, progesterone is often given to the patient as part of treatment and in the case of the PR-imaging agent, the technology would possibly be both therapeutic and diagnostic (theranostic).

Progesterone agents have been developed for positron emission topography (PET) imaging with success in targeting breast cancer cells and tissues in rat models (Zhou et al. J Med Chem 2006, 49, 4737-44., Vijaykumar et al. A. J Org Chem 2002, 67, 4904-10., Pomper et al. J Med Chem 1988, 31, 1360-3., herein incorporated by reference in their entireties). Metabolic conversion of reported progestin based PET agents prevented the application of these probes in humans (Dehdashti et al. J Nucl Med 1991, 32, 1532-7., herein incorporated by reference in its entirety).

In vivo imaging agents could provide a tool for basic scientific investigations into the etiology of disease by providing size and molecular profiles of tumors without the need to euthanize the animal. Mouse models of cancer and uterine tumors are an essential component of understanding how to prevent and treat disease. Many models could be improved by applying imaging techniques such that tumors could develop and differentiate without the need to remove the tumor mass directly.

For example, models of ductal carcinoma in situ (DCIS) are available, but understanding when the lesion forms, where, and whether it is steroid responsive is difficult to accomplish without sacrificing the animal and performing a dissection. However, if contrast agents were applied, small lesions could be identified very early, allowing one to determine if they form invasive cancers, and then categorizing the cancer as steroid responsive or unresponsive. Progesterone receptor positive breast and uterine cancer cells can be subcutaneously injected into nude mice and produce solid tumors monitored with magnetic resonance imaging (Zong et al. Magn Reson Med 2005, 53, 835-42., Preda et al. J Magn Reson Imaging 2004, 20, 865-73., herein incorporated by reference in their entireties). The breast cancer cell lines typically used for such tumors include T47D and MCF7 cell lines, both of which express estrogen receptor (ER) and progesterone receptor (PR) (Hoffmann et al. J Natl Cancer Inst 2004, 96, 210-8., herein incorporated by reference in its entirety). For uterine cancers, the Ishikawa cell line lacks receptors and stable clones of the cell line with the PR gene integrated allow the investigator to analyze both receptor positive and negative tumors. Xenografted tumors visibly protrude from the mouse but may be analyzed earlier and with more ease using MR imaging (Bhujwalla et al. Neoplasia 2001, 3, 143-53., herein incorporated by reference in its entirety). These nude mouse tumor models will be used to study the targeting ability of progesterone based contrast agents to image PR+ receptor positive tumors and for quantitative imaging to determine tumor response to drug therapies.

### SUMMARY

In some embodiments, the present invention provides a composition comprising: a) a ligand moiety, b) a contrast moiety, and c) a linkage region, wherein the linkage region covalently links the ligand moiety to the contrast moiety. In some embodiments, the ligand moiety comprises a hormone. In particular embodiments, the ligand moiety is produced naturally in a human or animal. In certain embodiments, the ligand moiety is a natural, non-synthetic hormone. In some embodiments, the hormone comprises progesterone. In some embodiments, the contrast moiety comprises a metal-ion chelator. In some embodiments, the metal-ion chelator comprises DO3A. In some embodiments, the metal-ion chelator coordinates a paramagnetic metal ion. In some embodiments, the paramagnetic metal ion includes, but is not limited to Gd(III), Fe(III), Mn(II), Y(III), Cr(III), Eu(III), and Dy(III). In some embodiments, the metal-ion chelator coordinates Gd(III). In some embodiments, the linker region comprises one or more methylene carbons. In some embodiments, the linker region comprises 3 or 6 methylene carbons. In some embodiments, the linker region comprises a covalent bond between the contrast moiety and the ligand moiety.

In some embodiments, the present invention provides a composition comprising: a) a hormone, b) metal-ion chelator, and c) a linkage region, wherein the linkage region covalently links the hormone to the metal-ion chelator. In some embodiments, the hormone comprises progesterone. In some embodiments, the metal-ion chelator coordinates a paramagnetic metal ion. In some embodiments, the paramagnetic metal ion comprises Gd(III).

In some embodiments, the present invention provides a method comprising: a) administering a composition comprising: a) a ligand moiety (e.g. progesterone), b) a metal-ion chelator (e.g. which chelates Gd(III)), and c) a linkage region, wherein the linkage region covalently links the ligand moiety (e.g. progesterone) to the metal-ion chelator to a cell, tissue, or patient, and b) producing a magnetic resonance image of the cell, tissue or patient. In some embodiments, the cell expresses progesterone receptor. In some embodiments, the progesterone binds to a progesterone receptor. In some embodiments, the binding of the progesterone to the progesterone receptor results in localization of the composition. In some embodiments, the cells do not express progesterone receptor.

### BRIEF DESCRIPTION OF THE DRAWINGS

The foregoing summary and detailed description is better understood when read in conjunction with the accompanying drawings which are included by way of example and not by way of limitation.

FIG. 1 shows structures of exemplary progesterone-conjugated MRI contrast agents.

FIG. 2 shows an exemplary synthesis scheme for progesterone modified Gd(III) chelate conjugates: (A) synthesis of neutral conjugates containing no spacer between the contrast moiety and the ligand moiety, (B) synthesis of neutral conjugates containing with a six-carbon linker region between the contrast moiety and the ligand, and (C) synthesis of charged progesterone conjugates.

FIG. 3 shows cellular uptake studies of progesterone-modified contrast agents: (A) progesterone-Gd(III) chelates are dose dependently absorbed into mammalian breast cancer cells that either express progesterone or are receptor negative, (B) progesterone-Gd(III) chelates are time dependently absorbed into mammalian breast cancer cells that either express progesterone or are receptor negative, and (C) progesterone-Gd(III) chelates are selectively retained in progesterone receptor-expressing cells at specific time points after leaching into culture medium.

FIG. 4 shows a graph demonstrating that progesterone-Gd(III) chelates function biologically to initiate gene transcription of a progesterone responsive element.

FIG. 5 shows in vitro MRI results: (A) T₁-weighted averages of breast cancer cells incubated with compounds 1 and 2 for 24 hours, and (B) T₁ data of cells incubated with 50, 150, and 500 μM of compound 1 or 2.

FIG. 6 shows chemical structures and names of steroid contrast agents.

FIG. 7 shows a graph demonstrating progesterone-Gd(III) chelates are selectively retained in progesterone receptor expressing cells. Gd(III) was quantified using ICP-MS.

FIG. 8 shows a graph demonstrating transcriptional upregulation of progesterone responsive promoter in response to PR-Gd(III) contrast agents.

FIG. 9 shows T₁-weighted images and measured spin-lattice relaxation times of cells incubated with compounds 1 and 2: Top Panel, MRI images acquired from PR-Gd(III) contrast agents inside of human breast cancer cells expressing the progesterone receptor; Bottom Panel, T₁ weighted values of mammary cells incubated with PR-Gd(III) contrast agents. The values are also depicted as the percent decrease in relaxivity as compare to the solvent control.

FIG. 10 shows mouse ovaries imaged before and after contrast agent 2 was injected. The arrows depict the ovary on either the left (A, C) or right (B, D) side of the body. The top two panels (A, B) were taken before injection and the bottom two panels were taken 30 minutes after 0.15 mmol/kg compound 2 was injected. Note the specific accumulation in the ovary, rich in progesterone receptors, but not in the kidney or bladder, organs that express low levels of PR.

FIG. 11 shows a histogram of the percentage of Gd(III) recovered per gram of various tissues for three Gd(III) compounds (2 and 24 hour time points).

FIG. 12 shows a histogram of the percentage of Gd(III) recovered per gram of various tissues for three Gd(III) compounds (30 min, 1 hour, 2 hour, 6 hour, and 24 hour time points).

### DEFINITIONS

As used herein, the term “sample” is used in its broadest sense. In one sense, it is meant to include a specimen or culture obtained from any source, as well as clinical, research, biological and environmental samples. Biological samples may be obtained from animals (including humans) and encompass cells, fluids, solids, tissues, and organs, and whole organisms. Such examples are not however to be construed as limiting the sample types applicable to the present invention.

The term “subject” refers to a cell, tissue, organ, animal, mammal, human, rodent, primate, etc. In some embodiments, the subject is a patient.

The term “bioactive molecule” refers to any chemical entity, whether in the solid, liquid, or gaseous phase which is capable of providing a biological effect when administered to a subject in accordance with the invention. The term “bioactive molecule” includes synthetic compounds, natural products and macromolecular entities such as polypeptides, polynucleotides, or lipids and also small entities such as neurotransmitters, ligands, hormones or elemental compounds. The term also includes such compounds whether in a crude mixture or purified and isolated.

As used herein, the term “relaxation time” refers to the time required for a nucleus which has undergone a transition into a higher energy state to return to the energy state from which it was initially excited. Regarding bulk phenomena, the term “relaxation time” refers to the time required for a sample of nuclei, the Boltzmann distribution of which has been perturbed by the application of energy, to reestablish the Boltzmann distribution. The relaxation times are commonly denoted T₁ and T₂. T₁ is referred to as the longitudinal relaxation time and T₂ is referred to as the transverse relaxation time. Other relaxation times of relevance include, but are not limited to T₁p (the paramagnetic contribution to the longitudinal relaxation rate) and T₂* (the transverse relaxation time including the effect of B₀ inhomogeneity). As used herein, the term “relaxation time” refers to the above-described relaxation times either together or in the alternative. Other relevant relaxation times will be apparent to those of skill in the art. An exhaustive treatise on nuclear relaxation is available in Banci, L, et al. NUCLEAR AND ELECTRON RELAXATION, VCH, Weinheim, 1991, which is herein incorporated by reference.

As used herein, the term “diagnostically effective amount” refers to an amount of contrast agent that is sufficient to enable imaging of the contrast agent in cells, tissues, or organisms using imaging equipment.

As used herein, the term “ligand” refers to is a molecule or molecules that are able to bind to and form a complex with a biomolecule.

### DETAILED DESCRIPTION

In some embodiments, the present invention provides targeted contrast agents for use in magnetic resonance imaging. In particular, the present invention provides contrast agents for magnetic resonance imaging prepared by conjugating a hormone (e.g. progesterone) to a metal-ion chelator (e.g. Gd(III) chelator) via a linker region. The contrast agents are cell permeable and accumulate in target cells based on the affinity for the hormone for its hormone receptor target. The metal-ion chelator provides a T₁ contrast agent capable of enhancing MRI signal in the region of contrast agent accumulation

Magnetic resonance imaging (MRI) has become an important tool in the clinical diagnosis of cancer. MRI provides noninvasive imaging of opaque specimens due to its high spatial and temporal resolution. The intrinsic magnetic resonance signal can be enhanced through the use of targeted contrast agents. Exogenous contrast agents manipulate relaxation times (T₁ and T₂) of water protons within a sample and enhance contrast in the image (U.S. Pat. No. 7,354,568; U.S. Pat. No. 7,029,655 ; U.S. Pat. No. 6,770,261; U.S. Pat. No. 6,713,046; U.S. Pat. No. 6,713,045; U.S. Pat. No. 6,656,450; U.S. Pat. No. 5,980,862; U.S. Pat. No. 5,707,605; Pub. App. No. 20060088475; Pub. App. No. 20050002866; Pub. App. No. 20040170563; Pub. App. No. 20030198597; Pub. App. No. 20030135108; Pub. App. No. 20030053954; Pub. App. No. 20030021750; Pub. App. No. 20030004236; Pub. App. No. 20020197648; Pub. App. No. 20020098153; and Pub. App. No. 20020049308; herein incorporated by reference in their entireties.

MRI images are acquired by employing radio frequency pulses to excite nuclear spins of a specimen and imposing one or more orthogonal magnetic field gradients (Merbach & Toth (2001).The Chemistry of Contrast Agents in Medical Magnetic Resonance Imaging, New York: John Wiley and Sons., Webb (1993) The Physics of Medical Imaging, Bristol, UK and Philadelphia: Institute of Physics Publishing., herein incorporated by reference in their entireties). The observed signal is that of the protons of water molecules, where signal intensity in a given volume element is a function of the water concentration and relaxation times (T₁ and T₂) (Allen & Meade (2004) Metal Ions in Biological Systems, Volume 42, New York: Fontis Media., herein incorporated by reference in its entirety). Optical microscopy of opaque specimens is limited by light scattering, whereas MRI can image in three dimensions with high spatial and temporal resolution (Meade et al. (2003) Curr. Opin. Neurobiol. 13, 597-602., Jacobs & Cherry (2001). Curr. Opin. Neurobiol. 11, 621-629., herein incorporated by reference in its entirety). Intrinsic MR contrast can be enhanced by using agents that modulate the spin-lattice relaxation rates of water protons (Caravan et al. (1999) Chem. Rev. 99, 2293-2352., herein incorporated by reference in its entirety). Paramagnetic ions can be used to decrease the local T₁ relaxation, and when chelated are nontoxic contrast agents.

A barrier to the development of MRI as a diagnostic tool has been the lack of targeted contrast agents. Previous approaches to develop targeted contrast agents have been limited by the ability of the agent to be linked to an antibody without perturbing the recognition properties or limited by the amount of uptake into cells (Louie et al. (2000) Nat. Biotechnol. 18, 321-325., Li et al. (2002) Inorg. Chem. 41, 4018-4024., Duimstra et al. (2005) J. Am. Chem. Soc. 127, 12847-12855., Allen et al. (2004) Chem. Biol. 11, 301-307., Artemov et al. (2003) Cancer Res. 63, 2723-2727., herein incorporated by reference in their entireties). Therefore, targeted strategies for new generations of chelates and ligands are required to improve cellular permeability and specificity of MRI agents.

The progesterone receptor (PR) is a member of the nuclear receptor superfamily that functions as a ligand activated transcription factor. Mammary epithelial cells express the PR and estrogen receptor (ER) (Ismail et al. (2003) Steroids 68, 779-787., herein incorporated by reference in its entirety). The PR is present in two distinct isoforms both derived from the same gene, PRA and PRB. Each subtype is critical to mammary gland lobuloalveolar development and epithelial differentiation (Lanari & Molinolo (2002) Breast Cancer Res. 4, 240-243., herein incorporated by reference in its entirety). The receptor consists of several regions that serve as functional units such as the DNA binding domain, the ligand binding domain, and transcriptional activation domains. The expression of these receptors is a parameter typically examined using immunohistochemistry in biopsies of human breast cancers (Jacobsen et al. (2003) J. Mammary Gland Biol. Neoplasia 8, 257-268., Bardou et al. (2003) J. Clin. Oncol. 21, 1973-1979., herein incorporated by reference in their entireties). The presence of both receptors correlates with the survival rate of breast cancer patients (Hopp (2004) Clin. Cancer Res. 10, 2751-2760., herein incorporated by reference in its entirety). The PR is an estrogen-regulated gene that becomes activated and expressed in the presence of estradiol and ER. Treatment with tamoxifen reduces PR and correlates with tamoxifen resistance, although the mechanism of resistance is still debated (Arpino et al. (2005) J. Natl. Cancer Inst. 97, 1254-1261., herein incorporated by reference in its entirety). Tumors that are ER+/PR− are considered more aggressive than PR+ tumors and correlate with a lower survival rate (Cui et al. (2005) J. Clin. Oncol. 23, 7721-7735., Muss (1992) Breast Cancer Res. Treat. 21, 15-26., herein incorporated by reference in its entirety). The current clinical methods to determine PR and ER levels require tissue biopsy or radioisotope injection followed by ionizing radiation. Therefore, noninvasively determining whether or not a mammary cancer expresses PR may be crucial to deciding the most effective chemotherapeutic agent for the patient.

As a result of its hydrophilic nature, many MR contrast agents using Gd(III) chelates do not traverse cell membranes and are restricted to the extracellular domains (Allen and Meade (2003). J. Biol. Inorg. Chem. 8, 746-750., Allen et al. (2004) Chem. Biol. 11, 301-307., herein incorporated by reference in their entirety). These chelates are thermodynamically stable (Kd=10²¹˜10²⁵) and kinetically inert. Embodiments of the present invention provide imaging systems employing steroids to facilitate cell entry of contrast agents. Steroids readily diffuse across the phospholipid bilayer due to their hydrophobic properties and small size (Rao (1981) Mol. Cell. Endocrinol. 21, 97-108., herein incorporated by reference in its entirety). Further, the availability of receptor-specific hormones provides a basis for determining whether the steroid is retained within a specific cell type, allowing the cell, and not the contrast agent, to determine molecular targeting. The steroid progesterone is an endogenous molecule with limited toxic activity and well-established pharmacokinetic profiling (Golub et al. (2006). Birth Defects Res. B Dev. Reprod. Toxicol. 77, 455-470., herein incorporated by reference in its entirety). In addition, steroids are typically retained in the nucleus of cells once bound to their receptor, where they interact with the DNA to drive gene transcription. Contrast agents that bind to large macromolecules such as enzymes or proteins undergo a dramatic increase in the relaxation rate of nearby water protons (Merbach & Toth (2001). The Chemistry of Contrast Agents in Medical Magnetic Resonance Imaging, New York: John Wiley and Sons., Webb (1993) The Physics of Medical Imaging, Bristol, UK and Philadelphia: Institute of Physics Publishing., herein incorporated by reference in their entireties). Binding to a macromolecule increases concentration and retention of the Gd(III) complex at the receptor binding site and affords an increase in rotational correlation time (τᵣ) of the agent.

As breast tumors become more aggressive, they typically lose hormone receptors and become less responsive to hormone-based breast cancer therapies. Few methods are available for imaging breast tumors in vivo, and none facilitate the molecular or therapeutic profiling of tumors. Previously, RU-486-modified MR contrast agents were proved to be membrane permeable, and successfully interact with progesterone receptors in cells and provide limited signal enhancement. Experiments were conducted during the development of embodiments of the invention to develop Gd(III)-conjugated steroid contrast agents that accumulate intracellularly, where they interact with nuclear progesterone receptors and thereby increase magnetic resonance. By using a progesterone receptor targeted contrast agent, it was contemplated that one can visualize the morphology of organs and diseased tissue and to determine the biochemical characteristics of cells. Embodiments of the present invention also provide synthetic methodologies for preparing new MRI contrast agents that are targeted to other hormones for imaging of hormone-dependent cancers.

In some embodiments the present invention provides a composition comprising a) a ligand moiety, b) a contrast moiety (e.g. metal-ion chelator and metal ion), and c) a linkage region, wherein the linkage region covalently links the ligand to the metal-ion chelator. In some embodiments the ligand moiety is a hormone, progesterone, a progesterone receptor-specific ligand, or anti-progesterone receptor antibody. In some embodiments, the contrast moiety comprises a metal-ion chelator and a metal ion. In some embodiments the metal-ion chelator of the contrast moiety comprises diethylenetriaminepentaacetic acid (DTPA), substituted DTPA, 1,4,7,10-tetraazacyclododecaneN,N′,N″, N′″-tetraacetic acid (DOTA), substituted DOTA, or other suitable chelators described in U.S. Pat. Nos. 5,155,215, 5,087,440, 5,219,553, 5,188,816, 4,885,363, 5,358,704, 5,262,532, and Meyer et al., Invest. Radiol. 25:S53 (1990), among others. In some embodiments generally suitable linkage regions include, but are not limited to, alkyl and aryl groups, including substituted alkyl and aryl groups and heteroalkyl (particularly oxo groups) and heteroaryl groups, including alkyl amine groups. In some embodiments the present invention provides a method comprising administering such a composition to a cell, tissue or patient. In some embodiments the method further comprises producing a magnetic resonance image of said cell, tissue or patient. In some embodiments the method is used for diagnostic or research purposes (e.g. drug screening applications).

In some embodiments, the present invention provides a contrast moiety which is configured to manipulate the relaxation times of surrounding water proton spins. In some embodiments, contrast moiety is configured to manipulate the longitudinal (T₁) and/or transverse (T₂) relaxation times. In some embodiments, contrast moiety is configured to manipulate T₁ relaxation of surrounding protons. In some embodiments, the materials of the present invention comprise one or more T₁ contrast agents. In some embodiments, T₁ contrast agents cause a reduction in the T₁ relaxation (e.g. increased relaxation time, decreased relaxation rate) resulting in increased signal intensity on T₁ weighted images. In some embodiments T₁ contrast agents are known as positive contrast agents. In some embodiments, T₁ contrast agents are small molecular weight compounds. In some embodiments, the contrast moiety of the present invention comprises a metal-ion chelator. In some embodiments, the contrast moiety of the present invention comprises a metal-ion chelator and a paramagnetic metal ion. In some embodiments, a paramagnetic metal ion is chelated by a metal-ion chelator of the contrast moiety. In some embodiments, T₁ contrast agents contain a paramagnetic metal ion as the active element of the paramagnetic contrast agents. Exemplary paramagnetic contrast agents suitable for use in the present compositions include, for example, stable free radicals, such as, for example, stable nitroxides, as well as compounds comprising transition, lanthanide and actinide elements, which may, if desired, be in the form of a salt or may be covalently or non-covalently bound to complexing agents, including lipophilic derivatives thereof, or to polypeptide-containing macromolecules. Preferable transition, lanthanide and actinide elements include, for example, Gd(III), Mn(II), Cu(II), Cr(III), Fe(II), Fe(III), Co(II), Er(II), Ni(II), Eu(III) and Dy(III). The foregoing elements may, if desired, be in the form of a salt, including inorganic and organic salts. In some embodiments, the contrast moiety comprises gadolinium (e.g. Gd(III)).

These elements may also, if desired, be complexed, for example, through covalent or noncovalent association, to one or more complexing agents, including metal-ion chelators, lipophilic derivatives, or to polypeptide-containing macromolecules. Preferable complexing agents for the present invention include, for example, diethylenetriaminepentaacetic acid (DTPA), ethylene-diaminetetraacetic acid (EDTA), 1,4,7,10-tetraazacyclododecane-N,N′,N″,N′″-tetraacetic acid (DOTA), 1,4,7,10-tetraazacyclododecane-N,N′,N″-triacetic acid (DOTA), 3,6,9-triaza-12-oxa-3,6,9-tricarboxymethylene-10-carboxy-13-phenyl-tridecanoic acid (B-19036), hydroxybenzylethylenediamine diacetic acid (HBED), N,N′-bis(pyridoxyl-5-phosphate)ethylene diamine, N,N′-diacetate (DPDP), 1,4,7-triazacyclononane-N,N′,N″-triacetic acid (NOTA), 1,4,8,11-tetraazacyclotetradecane-N,N′,N″,N′″-tetraacetic acid (TETA), kryptands (macrocyclic complexes), and desferrioxamine. More preferably, the complexing agents are EDTA, DTPA, DOTA, DO3A and kryptands, most preferably DTPA. Preferable lipophilic complexes include alkylated derivatives of the complexing agents EDTA, DOTA, for example, N,N′-bis(carboxydecylamidomethyl-N-2,3-dihydroxypropyl)-ethylenediamine-N,N′-diacetate (EDTA-DDP); N,N′-bis-(carboxy-octadecylamido-methyl-N-2,3-dihydroxypropyl)-ethylenediamine-N,N′-diacetate (EDTA-ODP); N,N′-Bis(carboxy-laurylamidomethyl-N-2,3-dihydroxypropyl)ethylenediamine-N,N′-diacetate (EDTA-LDP); and the like, including those described in U.S. Pat. No. 5,312,617, the disclosures of which are hereby incorporated herein by reference, in their entirety. Preferable polypeptide-containing macromolecules include, for example, albumin, collagen, polyarginine, polylysine, polyhistidine, gamma-globulin and beta-globulin, or any polypeptide sequence.

Suitable complexes therefore include, and may be of the type of, but are not limited to: Mn(II)-DTPA, Mn(II)-EDTA, Mn(II)-DOTA, Mn(II)-DO3A, Mn(II)-kryptands, Gd(III)-DTPA, Gd(III)-DOTA, Gd(III)-DO3A, Gd(III)-kryptands, Cr(III)-EDTA, Cu(II)-EDTA, or iron-desferrioxamine.

Additionally, the present invention may utilize a number of different magnetic resonance contrast agents that are well known in the art, and are disclosed in, for example, U.S. Pat. Nos. 5,141,740; 5,078,986; 5,055,288; 5,010,191; 4,826,673; 4,822,594; and 4,770,183, which are incorporated herein by reference. Such magnetic resonance contrast agents include many different paramagnetic contrast agents, for example, gadolinium compounds.

In some embodiments, the present invention provides a linker region (e.g. a region which connects a contrast moiety and a ligand moiety). In some embodiments of the present invention, the ligand moiety and contrast moiety are linked, either directly (e.g. linker region comprises a covalent bond) or linked via a suitable linker (e.g. linker region comprises a linker group). The present invention is not limited to any particular linker group. Indeed, a variety of linker groups are contemplated, suitable linkers could comprise, but are not limited to, alkyl groups, methylene carbon chains, ether, polyether, alkyl amide linker, a peptide linker, a modified peptide linker, a Poly(ethylene glycol) (PEG) linker, a streptavidin-biotin or avidin-biotin linker, polyaminoacids (e.g. polylysine), functionalised PEG, polysaccharides, glycosaminoglycans, dendritic polymers (WO93/06868 and by Tomalia et al. in Angew. Chem. Int. Ed. Engl. 29:138-175 (1990), herein incorporated by reference in their entireties), PEG-chelant polymers (W94/08629, WO94/09056 and WO96/26754, herein incorporated by reference in their entireties), oligonucleotide linker, phospholipid derivatives, alkenyl chains, alkynyl chains, disulfide, or a combination thereof.

A targeted contrast agent of the present invention may comprise a single linker region or multiple linker regions (e.g. 1 linker, 2 linkers, 3 linkers, 4 linkers 5 linkers . . . 10 linkers . . . 20 liners, etc.). In some embodiments the linker comprises a single chain connecting one ligand moiety to one contrast moiety. In some embodiments, there are multiple linkers connecting multiple ligand moieties to a single contrast moiety. In some embodiments, a linker may connect multiple ligand moieties to each other. In some embodiments, a linker may connect multiple contrast moieties to each other. In some embodiments, a linker attaches an additional functional portion to a ligand moiety and/or contrast moiety. In some embodiments, a linker may be branched, connecting more than two ligand moieties and/or contrast moieties. A linker may be flexible, or rigid. A linker may be of any suitable length, and contain any suitable number of atoms and/or subunits.

In some embodiments, the linker of the present invention is cleavable or selectively cleavable. In some embodiments, the linker is cleavable under at least one set of conditions, while not being substantially cleaved (e.g. approximately 50%, 60%, 70%, 80%, 90%, 95%, 99%, or greater remains uncleaved) under another set (or other sets) of conditions. In some embodiments, the linker is susceptible to cleavage under specific conditions relating to pH, temperature, oxidation, reduction, UV exposure, exposure to radical oxygen species, chemical exposure, light exposure (e.g. photo-cleavable), etc.

In some embodiments, the linker region is photocleavable. That is, upon exposure to a certain wavelength of light, the linker region is cleaved, allowing release of the connected contrast agents. This embodiment has particular use in developmental biology fields (cell lineage, neuronal development, etc.), where the ability to follow the fates of particular cells is desirable. A particularly preferred class of photocleavable linkers are the O-nitrobenzylic compounds, which can be synthetically incorporated via an ether, thioether, ester (including phosphate esters), amine or similar linkage to a heteroatom (particularly oxygen, nitrogen or sulfur). Also of use are benzoin-based photocleavable linkers. A wide variety of suitable photocleavable moieties is outlined in the Molecular Probes Catalog, supra.

In some embodiments, the linker is susceptible to enzymatic cleavage (e.g. proteolysis). In some embodiments of the present invention, the ligand moiety and contrast moiety are linked, via a cleavable linker. The present invention is not limited to any particular linker group. In some embodiments, the cleavable linker region contains a peptide portion. In some embodiments, the peptide portion of the cleavable linker region is cleavable. In some embodiments, the peptide portion of the cleavable linker region is enzymatically cleavable. In some embodiments, the peptide portion of the cleavable linker region is configured to be cleaved by proteolysis. In some embodiments the cleavable linked contains a specific proteolytic site.

In some embodiments, multiple linker regions are contemplated. A linker region comprising a complex linker constructed from a variety of linker groups is contemplated. Suitable linker groups for construction of a complex linker may comprise, but are not limited to, alkyl groups, methylene carbon chains, ether, polyether, alkyl amide linker, a peptide linker, a modified peptide linker, a Poly(ethylene glycol) (PEG) linker, a streptavidin-biotin or avidin-biotin linker, polyaminoacids (eg. polylysine), functionalised PEG, polysaccharides, glycosaminoglycans, dendritic polymers (WO93/06868 and by Tomalia et al. in Angew. Chem. Int. Ed. Engl. 29:138-175 (1990), herein incorporated by reference in their entireties), PEG-chelant polymers (W94/08629, WO94/09056 and WO96/26754, herein incorporated by reference in their entireties), oligonucleotide linker, phospholipid derivatives, alkenyl chains, alkynyl chains, disulfide, or a combination thereof.

In some embodiments, the present invention provides a ligand moiety. In some embodiments, the ligand moiety comprises a small molecule which is configured to bind to or be bound by another molecule (e.g. a binding partner). In some embodiments, the ligand moiety interacts with one or more binding partners through non-covalent binding, covalent binding, hydrogen binding, van der Waals forces, ionic bonds, hydrophobic interactions, electrostatic interaction, and/or combinations thereof In some embodiments, the ligand moiety may comprise synthetic compounds, natural products, macromolecular entities such as polypeptides, polynucleotides or lipids, and also small entities such as neurotransmitters, substrates, ligands, small drug-like molecules, hormones or elemental compounds. In some embodiments, the ligand moiety comprises a hormone, hormone derivative, or hormone-like molecule. In some embodiments, the ligand moiety comprises a hormone selected from the list of melatonin, serotonin, thyroxine, triiodothyronine, epinephrine, norepinephrine, dopamine, antimullerian hormone, adiponectin, adrenocorticotropic hormone, angiotensinogen, angiotensin, antidiuretic hormone, vasopressin, arginine vasopressin, corticotrophin, atrial-natriuretic peptide, atriopeptin, calcitonin, cholecystokinin, corticotropin-releasing hormone, erythropoietin, follicle-stimulating hormone, gastrin, ghrelin, glucagon, gonadotropin-releasing hormone, growth hormone-releasing hormone, human chorionic gonadotropin, human placental lactogen, growth hormone, inhibin, insulin, insulin-like growth factor, somatomedin, leptin, luteinizing hormone, melanocyte stimulating hormone, orexin, oxytocin, parathyroid hormone, prolactin, relaxin, secretin, somatostatin, thrombopoietin, thyroid-stimulating hormone, thyrotropin, thyrotropin-releasing hormone, cortisol, aldosterone, testosterone, dehydroepiandrosterone, androstenedione, dihydrotestosterone, estradiol, estrone, estriol, progesterone, calcitriol, calcidiol, prostaglandins, leukotrienes, prostacyclin, thromboxane, prolactin releasing hormone, lipotropin, brain natriuretic peptide, neuropeptide Y, histamine, endothelin, pancreatic polypeptide, renin, enkephalin, human variations thereof, non-human variations thereof, combinations thereof, derivatives thereof, etc. In some embodiments, the ligand moiety comprises a steroid hormone selected from the classes of progestagens, estrogens, androgens, mineralocorticoids, and glucocorticoids. In some embodiments the ligand moiety is a hormone, progesterone, a progesterone receptor-specific ligand, or anti-progesterone receptor antibody. In some embodiments, the ligand moiety comprises progesterone.

In some embodiments, the ligand moiety is configured to bind to a biologically relevant molecule, biomolecule, or biological molecular complex. In some embodiments, a ligand moiety of the present invention binds to a protein, peptide, polypeptide, antibody, receptor protein, nucleic acid (e.g. RNA, DNA), carbohydrate, lipid, macromolecule, macromolecular complex, complex thereof, combination thereof, etc. In some embodiments, the ligand moiety is configured to bind to a receptor protein (e.g. hormone receptor (e.g. steroid hormone receptor (e.g. progesterone receptor))). In some embodiments, the ligand moiety is configured to bind to a steroid hormone receptor selected, for example, from the classes of type-I receptors, sex hormone receptors, androgen receptor, estrogen receptor, progesterone receptor, glucocorticoid receptor, mineralocorticoid receptors, type-II receptors, vitamin A receptor, vitamin A receptor, retinoid receptor, thyroid hormone receptor, and the like.

In some embodiments, the ligand moiety (e.g. a small molecule ligand (e.g. a hormone (e.g. progesterone))) is configured to bind to a biomolecule (e.g. macromolecule, protein (e.g. antibody, receptor (e.g. hormone receptor (e.g. progesterone receptor)))). In some embodiments, binding of the ligand moiety (e.g. a small molecule ligand (e.g. a hormone (e.g. progesterone))) to a biomolecule (e.g. macromolecule, protein (e.g. antibody, receptor (e.g. hormone receptor (e.g. progesterone receptor)))) results in targeting the contrast agent complex of the present invention to the specific biomolecule. In some embodiments, the binding of the ligand moiety to a biomolecule of interest results in co-localization of the contrast moiety and the molecule of interest. In some embodiments, binding of the ligand moiety to the biomolecule of interest provides enhanced imaging of the biomolecule of interest as a result of the co-localized contrast moiety. In some embodiments, the ligand moiety targets the contrast moiety to a biomolecule of interest.

In some embodiments, the present invention provides an additional functional portion along with, or in place of, the ligand moiety, linker region, and/or contrast moiety. In some embodiments, an additional functional portion is an optical dye. In some embodiments, the additional functional portion is a chromophore. In some embodiments, an optical dye functional portion allows co-localization of optical imaging with MRI. In some embodiments, the present invention allows co-localization of the contrast moiety with an optical dye functional portion. In some embodiments, the optical dye is selected from the group including, but not limited to acridine dyes, anthraquinone dyes, arylmethan dyes, azo dyes, cyanine dyes, diazonium dyes, nitro dyes, nitroso dyes, phenaanthridine dyes, pthalocyanine dyes, quinine-imine dyes, indamins, indophenols dyes, oxazin dyes, oxazone dyes, thiazin dyes, thiazole dyes, xanthenes dyes, fluorene dyes, pyronin dyes, fluorine dyes, rhodamine dyes, etc. In some embodiments, the optical dye is a fluorophore selected from the list including, but not limited to (E)-stilbene, (Z)-Stilbene, 7-Amino-actinomycin D, Acridine orange, Acridine yellow, Alexa Fluor, Auramine O, Auramine-rhodamine stain, Benzanthrone, 9,10-Bis(phenylethynyl)anthracene, 5,12-Bis(phenylethynyl)naphthacene, CFDA-SE, CFSE, Calcein, Carboxyfluorescein, 1-Chloro-9,10-bis(phenylethynyl)anthracene, 2-Chloro-9,10-bis(phenylethynyl)anthracene, Coumarin, Cyanine, DAPI, Dark quencher, DiOC6, DyLight Fluor, Ethidium bromide, Fluorescein, Fura-2, Fura-2-acetoxymethyl ester, Green fluorescent protein (GFP) and modifications of GFP that have different absorption/emission properties, HiLyte Fluor, Hoechst stain, Indian yellow, Indo-1, Luciferin, Nile red, Perylene, Phycobilin, Phycoerythrin, Phycoerythrobilin, Propidium iodide, Pyranine, Rhodamine, RiboGreen, Rubrene, Ruthenium(II) tris(bathophenanthroline disulfonate), SYBR Green, Sulforhodamine 101, Sulforhodamine B, TSQ, Texas Red, Umbelliferone, and Yellow fluorescent protein.

In some embodiments, an additional functional portion is a biomolecule, such as for example, a ligand, antibody, peptide, polypeptide, protein, nucleic acid, polysaccharide, carbohydrate, lipid, glycoprotein, phosphlipid, sterol, hormone, disaccharide, amino acid, nucleotide, phosphate, monsacharide, etc. In some embodiments, a biomolecule functional portion serves to localize the imaging system in a specific cell type, for example, blastomere, embryonic stem cell, erythrocyte, fibroblast, hepatocyte, myoblast, myotube, neuron, oocyte, osteoblast, osteoclast, T-Cell, zygote, prokaryotic cell, a specific bacteria, plant cells, fungal cells, etc. In some embodiments, a biomolecule functional portion serves to localize the imaging system in a specific cellular region, for example cytoplasm, nucleus, intracellular space, golgi complex, endoplasmic reticulum, mitochondria, chloroplasts, etc. In some embodiments, a biomolecule functional portion serves to localize the imaging system in a specific tissue, for example, epithelial, connective, muscle, neural, etc. In some embodiments, a biomolecule functional portion serves to localize imaging system in specific diseased cells, for example, cancer cells, virally infected cells, etc. In some embodiments, a biomolecule functional portion serves to interact with native biomolecules in a subject, sample, tissue, or cell, such as for example, cell surface markers, antibodies, receptor proteins, nucleic acid, specific classes of proteins, etc.

In some embodiments, an additional functional portion is a biomolecule which serves as a targeting moiety. Herein, the term “targeting moiety” is meant a functional group which serves to target or direct the complex to a particular location, cell type, diseased tissue, or association. In general, the targeting moiety is directed against a target molecule.

In some embodiments, an additional functional portion is a tag allowing the imaging system to be used with additional imaging modalities. In some embodiments, an additional imaging modality provides co-localization of multiple imaging modalities. In some embodiments, an additional imaging modality provides co-localization of an additional imaging modality with the contrast moiety of the imaging system. In some embodiments, an additional functional portion allows the imaging system to be used with, for example, nuclear medicine, molecular imaging, positron emission tomography (PET), single photon emission computed tomography (SPECT), optical imaging, infrared imaging, fluoroscopy, angiography, computed tomography (CT) scanning, etc. In some embodiments, a tag may comprise an additional contrast moiety configured to enhance T₁ and/or T₂ relaxation.

In some embodiments, the present invention provides contrast agents to be used in generating an image of a cell, tissue, organ, patient, human subject, or non-human subject by administering the contrast agent to the subject (e.g. vascularly, via the gastrointestinal tract, etc.) and generating an image of at least a part of the subject to which the contrast agent has distributed.

In some embodiments, the present invention is used by administering a contrast agent of the present invention to a subject. Known methods for administering therapeutics and diagnostics can be used to administer contrast agents for practicing the present invention. For example, fluids that include pharmaceutically and physiologically acceptable fluids, including water, physiological saline, balanced salt solutions, buffers, aqueous dextrose, glycerol or the like as a vehicle, can be administered by any method used by those skilled in the art. These solutions are typically sterile and generally free of undesirable matter. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration and imaging modality selected. The invention further provides formulations comprising the contrast agent of the invention and a pharmaceutically acceptable excipient, wherein the contrast agent is formed according to any of the above described embodiments, and wherein the formulation is suitable for administration as an imaging enhancing agent and the contrast agent is present in an amount sufficient to enhance a magnetic resonance tomography image. These agents can be administered by any means in any appropriate formulation. Detergents can also be used to stabilize the composition or the increase or decrease the absorption of the composition. Other physiologically acceptable compounds include wetting agents, emulsifying agents, dispersing agents or preservatives that are particularly useful for preventing the growth or action of microorganisms. One skilled in the art appreciates that the choice of an acceptable carrier, including a physiologically acceptable compound depends, for example, on the route of administration and on the particular physio-chemical characteristics of any co-administered agent.

Methods of introduction include, but are not limited to, intradermal, intramuscular, intraperitoneal, intravenous, subcutaneous, intranasal, rectal, vaginal, and oral routes. The compositions may be administered by any convenient route, for example by infusion or bolus injection, by absorption through epithelial or mucocutaneous linings (e.g., oral mucosa, vaginal, rectal and intestinal mucosa, etc.) and may be administered together with other biologically active agents. Administration can be systemic or local. In addition, the contrast agent compositions may be introduced into the central nervous system by any suitable route, including intraventricular and intrathecal injection; intraventricular injection may be facilitated by an intraventricular catheter, for example, attached to a reservoir, such as an Ommaya reservoir. The compositions of the invention can be delivered by any means known in the art systematically (e.g. intra-venously), regionally or locally (e.g. intra- or peri-tumoral or intra-cystic injection, e.g. to image bladder cancer) by e.g. intra-arterial, intra-tumoral, intra-venous (iv), parenteral, intra-pneural cavity, topical, oral or local administration, as sub-cutaneous intra-zacheral (e.g. by aerosol) or transmucosal (e.g. voccal, bladder, vaginal, uterine, rectal, nasal, mucosal), intra-tumoral (e.g. transdermal application or local injection). For example, intra-arterial injections can be used to have a “regional effect”, e.g. to focus on a specific organ (e.g. brain, liver, spleen, lungs). For example intra-hepatic artery injection or intra-carotid artery injection may be used. If it is decided to deliver the preparation to the brain, it can be injected into a carotid artery or an artery of the carotid system of arteries (e.g. ocipital artery, auricular artery, temporal artery, cerebral artery, maxillary artery etc.). The present invention also provides pharmaceutical compositions which include contrast agents, alone or with a pharmaceutically acceptable carrier.

In some embodiments, amounts of the contrast agents sufficient to provide the desired results will be used, balanced by other considerations such as whether the contrast agent used for a particular application might produce undesirable physiological results. In some embodiments, the precise dose to be employed in the formulation can also depend on the route of administration, and should be decided according to the judgment of the practitioner and each subject's circumstances. In addition, in vitro and in vivo assays may optionally be employed to help identify optimal dosage ranges. Effective doses may be extrapolated from dose-response curves derived from in vitro or in vivo test systems. In some embodiments, the amounts of the contrast agent or agents administered can range from micromolar to molar amounts, but more likely will be used in millimolar-to-micromolar amounts.

The formulations of the invention can be administered in a variety of unit dosage forms, depending upon the particular cell or tissue or cancer to be imaged, the general medical condition of each patient, the method of administration, and the like. Details on dosages are well described on the scientific and patent literature. The exact amount and concentration of contrast agent or pharmaceutical of the invention and the amount of formulation in a given dose, or the “effective dose” can be routinely determined by, e.g. the clinician. The “dosing regimen” will depend upon a variety of factors, e.g. whether the cell or tissue or tumor to be imaged is disseminated or local, the general state of the patient's health, age and the like. Using guidelines describing alternative dosing regimens, e.g. from the use of other imaging contrast agents, the skilled artisan can determine by routine trials optimal effective concentrations of pharmaceutical compositions of the invention.

The present invention provides novel conjugate compositions comprising: a) a ligand moiety, b) a contrast moiety, and c) a linker region. In some embodiments, the present invention provides method of using such conjugates as contrast agents for MRI. In some embodiments, contrast agents of the present invention are administered to a sample, cell, tissue, or patient prior to magnetic resonance imaging of the sample, cell, tissue, subject, or patient. Contrast agents can be used in vivo or in vitro, and can be used in clinical, research, diagnostic, or treatment utilities. In some embodiments, compositions of the present invention may be administered to any cells prior to MRI, for example. In some embodiments, the present invention may find utility in vitro or in vivo applications. In some embodiments, compositions and methods of the present invention may be administered to a cell or tissue which has been isolated and/or purified. In some embodiments, compositions and methods of the present invention may be administered to a cell or tissue which is in the context of a subject or patient. In some embodiments, compositions and methods of the present invention may be administered to a patient or subject.

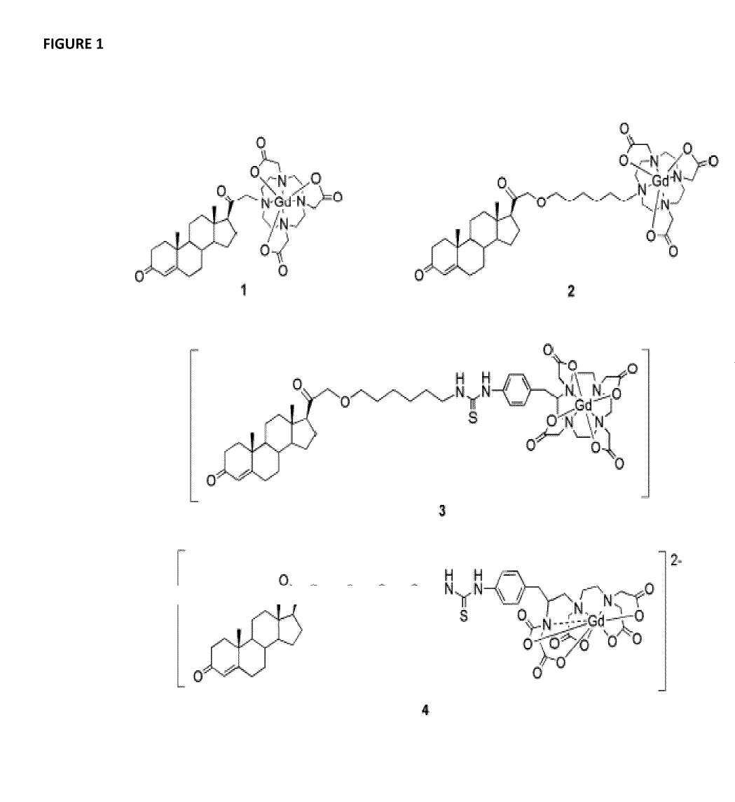

The present invention provides exemplary MR agents that were synthesized and evaluated to target progesterone receptors (SEE FIG. 1). Compound 2, demonstrated hormone receptor binding, progesterone-responsive gene transcription, and enhanced intracellular relaxivity. Thus, it is demonstrated that steroid receptor specific MR agents can be prepared and retain their ability to interact with their receptor to enhance relaxivity.

The progesterone-MR contrast agent conjugates were designed to optimize receptor interaction. A series of agents was synthesized with variable linkers between the Gd(III) chelate and the hormone backbone. The impact of linker length on receptor interaction was examined using progesterone receptor binding experiments. The agents with the highest affinity were those that had no spacer between the chelate and hormone, such as compound 1. Because it has a hydrophilic Gd(III) chelate instead of having a lipophilic chain on the 21 position, this might indicate that there is a favorable interaction between receptor protein and the chelate, although the present invention is not limited to any particular mechanism of action and an understanding of the mechanism of action is not necessary to practice the present invention. It is contemplated that altering the carbon chain with a hydrophilic PEG linker or poly-amino acid chain may result in higher receptor binding affinity. The progesterone Gd(III) chelates had an approximately 100-fold higher affinity for this receptor than the chelate conjugated to RU-486. Incorporating a linker region between the steroid and the chelate resulted in lower binding affinity for the receptor, perhaps through disruption of dimer formation, although the present invention is not limited to any particular mechanism of action and an understanding of the mechanism of action is not necessary to practice the present invention. The relatively tight binding of the hormone with the receptor demonstrates that the modification of the steroid decreases but does not prohibit interaction with the progesterone receptors.

Conjugation of a Gd(III) chelate to the steroid progesterone allowed for significant and rapid cellular accumulation. All of the compounds traversed the cell membrane in a dose-dependent manner. Conjugate 2 most readily entered the cells, as demonstrated by ICP-MS and X-ray analysis. Compound 1, however, was not as membrane permeable as the compounds containing a six-carbon extended spacer, likely due to its relatively low lipophilicity. The addition of different charges on the chelate adversely affected cell permeability. This may be due to the inability of the charged group to readily pass the hydrophobic membrane, although the present invention is not limited to any particular mechanism of action and an understanding of the mechanism of action is not necessary to practice the present invention. These experiments demonstrate that the steroid conjugation of the Gd(III) chelate allows it to be carried into the cell.

Progesterone-conjugated Gd(III) chelates are cell permeable and interact with the progesterone receptor, providing cell-specific image enhancement. Activation of the specific biological target by the contrast agent was directly demonstrated using the transcriptional activation of the PRE-luciferase construct. Transcriptional activation may enhance specific cellular targeting because the contrast agent would be engaged in a receptor:DNA complex and might be retained within the cell, although the present invention is not limited to any particular mechanism of action and an understanding of the mechanism of action is not necessary to practice the present invention. By directly comparing the amount of gadolinium that leached from progesterone receptor-expressing T47D cells as compared to receptor-negative MDA-MB-231 cells, a relative retention was calculated. Compound 2 was specifically retained in receptor-expressing cells. Compound 1 was not specifically retained, because the initial absorption of the compound is relatively low. Cellular retention is likely different between 1 and 2, due to the enhanced interaction with the receptor after high absorption and the generation of a transcription complex, although the present invention is not limited to any particular mechanism of action and an understanding of the mechanism of action is not necessary to practice the present invention. Evidence of transcriptional regulation is supported by the high level of luciferase activity generated by compound 2 in the PRE transcription assay. The conjugate-induced transcription provides evidence of cellular permeability, receptor-mediated retention, and a lack of toxicity. Cellular transcription suggests that these contrast agents may be useful for obtaining images long after traditional agents that are readily excreted.

Progesterone-based contrast agents provide magnetic resonance signal enhancement inside breast cancer cells. The T₁ effects of compound 2 were substantially changed at 150 mM in MDA-MB-231 cells both with and without receptor. Incubating the cells with higher doses of the contrast agent did not produce additional signal enhancement. Compound 1 was expected to produce a greater τᵣ effect because it has the highest binding affinity for the receptor and the slowest rotation of the Gd(III) chelate due to the absence of the six-carbon spacer. However, membrane permeability of 1 was significantly lower even at the highest concentration (500 mM) and failed to enhance MR contrast in vitro. Compound 2 showed high cellular accumulation; however, differences in the amount of accumulation inside cells between PR-positive and PR-negative were insignificant, thereby creating MR images with the same signal intensity. The changes in T₁ using relatively low doses of contrast agent indicate that these newly synthesized compounds are viable contrast agents that can be utilized in low doses.

A series of exemplary and illustrative progesterone-modified Gd(III) chelate conjugates were synthesized to generate contrast agents that accumulate intracellularly and interact with a biological target relevant for cancer prognosis. The chelates varied in linker length and charge, and compound 2 has been identified as an efficient agent for progesterone receptor binding and intracellular accumulation. The aliphatic carbon linker between the steroid and the Gd(III) chelate improved cellular permeability while retaining transcriptional activation of the progesterone responsive element without toxicity. Embodiments of the present invention provides that changing the modification site from 3-keto to 21-hydroxyl can enhance the binding affinity approximately 100-fold as compared to a previously reported RU-486-modified contrast agent. The observed relaxivity of the contrast agent in mammary cells was significant.

In some embodiments, the invention provides a kit comprising one or more containers filled with one or more of the contrast agent(s) compositions. In some embodiments, the compositions comprising the contrast agents of the present invention, may be packaged, stored, or administered in combination with other diagnostic or therapeutic treatments.

### EXPERIMENTAL

Examples 1 through 8 describe a set of experiments performed during development of embodiments of the present invention (Lee et al. (2007) Chemistry & Biology 14, 824-834., herein incorporated by reference in its entirety). Examples 9 through 14 describe a second set of experiments performed during the development of embodiments of the present invention.

### Example 1

### Compositions and Methods for Development and Characterization of MR-Contrast Agents

Octanol-Water Partition Coefficient Measurements. Octanol-water partition coefficients were obtained by dissolving 5-7 mg of each compound (1-4) into mixtures of 500 ml water and 500 ml 1-octanol. The resulting mixture was shaken vigorously for 2 hr on a LAB-LINE lab rotator (model 1304; LAB-LINE, Dubuque, Iowa, USA). The solvent layers were allowed to separate, and 400 ml of each layer was removed. The solvent was removed under reduced pressure, and the mass of material from each layer was measured. The reported values are for the mass of compound in the 1-octanol layer divided by the mass of compound in the water layer.

Relaxivity Measurements. Relaxivity measurements were acquired by taking the slope of a plot of T₁⁻¹ versus concentration. The longitudinal water proton relaxation time (T₁) at 59.97 MHz was measured using a BRUKER mq60 NMR analyzer (BRUKER Canada, Milton, ON, Canada). A 4 mM stock solution of each compound in deionized water was diluted to give 500 ml each of six concentrations for each run: 0, 0.125, 0.25, 0.5, 1.0, and 2.0 mM. For 3, a 1 mM stock solution in deionized water was diluted to give 250 ml each of six concentrations for each run: 0, 0.001, 0.005, 0.01, 0.05, and 0.1 mM. The T₁ of each concentration was determined using an inversion recovery pulse sequence with appropriate recycle delays. The resulting curves were fit to a monoexponential function to obtain T₁. Lines fit with r²>0.998.

Progesterone Receptor Binding Assay. The progesterone receptor A ligand binding domain (amino acids 675-933) fused to GST (PR-LBD-GST; 80 nM), a fluorescently tagged PR ligand (fluoromone green PL; 4 nM), and either progesterone (1 mM) or compound 1-4 (several concentrations) were incubated in PR screening buffer with 4 mM dithiothreitol (DTT) in a total volume of 100 ml for 1 hr at room temperature according to the manufacturer's protocol (INVITROGEN, Carlsbad, Calif., USA). A Beacon 2000 fluorescence polarization analyzer (INVITROGEN) was used to take fluorescence measurements. The machine was used in static mode, batch blank, no delay, with an average of 1 read per cycle, at 22° C. Buffer and PR-LBD-GST with no fluorescent PL was used as the blank to eliminate background signal from the protein or buffer. A sample with no competitor was used to determine 100% binding capacity of the PR-LBD-GST for the PL ligand.

Progesterone Response Element Transcriptional Activation. T47D breast cancer epithelial cells (American Type Culture Collection, Manassas, Va., USA) were cultured in phenol red-free RPMI (LIFE TECHNOLOGIES, Gaithersburg, Md., USA) supplemented with 10% fetal bovine serum (FBS) (INVITROGEN) and 1% antimycotic/antibiotic (INVITROGEN) and incubated at 37° C., under 5% CO₂. Cells were plated 1 day before transfection in 24-well plates and transiently transfected in Opti-MEM (INVITROGEN) with PRE-luciferase. Cells were then treated with serum-free media and vehicle (DMSO), progesterone, and progesterone-modified contrast agents for 24 hr. Cells were lysed in GME buffer (25 mM glycylglycine (pH 7.8), 15 mM MgSO4, 4 mM EGTA, 1 mM DTT, and 1% Triton X-100) and lysates were added to assay buffer (GME buffer, 16.5 mM KPO4, 2.2 mM ATP, and 1.1 mM DTT). Luciferase activity was measured for 30 seconds using an AUTOLUMAT (BERTHOLD TECHNOLOGIES, Oak Ridge, Tenn., USA). A separate protein determination using the BCA kit (PIERCE, Rockford, Ill., USA) was used to normalize protein levels that might differ from treatment with hormone.

Progesterone-Gd(III) Cellular Uptake. Progesterone receptor-positive cells, T47D, and progesterone receptor-negative cells (MDA-MB-23 1) were used to determine uptake efficiency of progesterone-modified contrast agents into hormone receptor-expressing cells. MDA-MB-231 breast cancer epithelial cells (American Type Culture Collection) were cultured in phenol red-free DMEM/F12 (LIFE TECHNOLOGIES) supplemented with 10 mg/ml insulin, 5% charcoal dextran-stripped FBS (CELLGRO, Herndon, Va., USA), 1% glutamax, and 1% antimycotic/antibiotic (INVITROGEN) and incubated at 37° C., under 5% CO2. Cells were plated into 12-well dishes and the next day they were moved into serum-free media for 24 hr before treatment with compound 1-4. The following doses were incubated with cells for 24 hr: 0, 0.05, 0.5, 5, and 50 mM. 50 mM PR-Gd compounds were incubated with the cells for the following time periods: 0, 1, 2, 4, and 24 hr. Data were analyzed by counting the cells, followed by lysis and ICP-MS. For leaching experiments, contrast agents were incubated with the cells for 4 hr, removed, and rinsed with PBS. At each time point after the initial rinse, cell media were removed, rinsed, and replaced with serum-free media and then collected at 15 min, 1, 2, 4, 6, 24, 48, and 72 hr. The cells were also collected by trypsinization and lysed to compare intracellular content of the cells with that which leached into the media.

Synchrotron Radiation X-Ray Fluorescence Analysis. The receptor-positive and -negative cells were incubated with 1 and 2 for 24 hr prior to analysis. The cells were washed and collected by following the same procedure as described above. Approximately 15 ml of each suspension was applied to Formvar-coated gold grids with a sterile glass Pasteur pipette for 1 min and the excess supernatant was removed. This was followed by addition of approximately 15 ml of room temperature ethanol, removal of the ethanol, and drying at ambient temperature for 15 hr. The cell coverage was approximately 15-30 cells/grid. Electron microscope grids with cells were mounted onto a kinematic specimen mount for both visible light and X-ray fluorescence microscopy. The samples were examined under a light microscope (LEICA DMXRE, Solms, Germany), and the cells to be scanned with SR-XRF were placed on the grid relative to a reference point using a high spatial resolution motorized x/y stage (LUDL BIOPRECISION, Hawthorne, N.Y., USA).

X-Ray Fluorescence Microscopy. Synchrotron scanning X-ray fluorescence microscopy was carried out at the 2-ID-E beamline of the Advanced Photon Source at Argonne National Laboratory (Argonne, Ill., USA). Hard X-rays (10 keV) from an undulator source were monochromatized using a single-bounce Si <111> monochromator. The energy was selected to allow for efficient excitation of the Gd L lines and to enable the detection of the Zn K lines. A Fresnel zone plate (320 mm diameter, focal length f=250 mm, Xradia, Concord, Calif., USA) was used to focus the monochromatic X-ray beam to a spot size of approximately 0.3×0.3 mm² on the specimen. The sample was raster scanned through the beam at room temperature under a helium atmosphere. At each scan position, a full fluorescence spectrum was acquired using an energy dispersive germanium detector (ULTRA-LEGE; Canberra, Meriden, Conn., USA). Elemental content was determined by comparison of fitted sample spectra with National Bureau of Standards thin film standards 1832 and 1833 (National Institute of Standards and Technology, Gaithersburg, Md., USA) using MAPS software supplemented with fitting of fluorescence spectra at each pixel.

T₁-Weighted Image Acquisition. The receptor-positive (T47D and MDA-MB-231 transfected with PRA) and -negative (MDA-MB-231) cells were incubated with no agent or 50, 150, and 500 mM compound 1 and 2 for 24 hr at 37° C. Cells were loaded into capillary tubes (1 mm diameter) as trypsin suspensions. MR data was collected at ambient temperature in a GENERAL ELECTRIC/BRUKER Omega 400WB 9.4 T magnet (83 mm bore size) fitted with ACCUSTAR shielded gradient coils (BRUKER, Westmont, Ill., USA). Spin lattice relaxation times (T₁) were measured using an inversion recovery pulse sequence, and images were acquired using a T₁-weighted spin-echo pulse sequence with a repetition time (T_{R}) of 100˜2000 ms and an echo time (T_{E}) of 10˜10.2 ms.

### Example 2

### PR-Targeted MR Contrast Agents

A series of progesterone conjugates with Gd(III) contrast agents was synthesized and characterized (FIG. 1). Previously, RU-486 was modified with a similar Gd(III) chelate, and it was discovered that the site of attachment was critical to the binding affinity of the complex. The RU-486-modified conjugate had approximately a 100-fold decrease in affinity for the receptor. The labeling strategy was modified for the synthesis of new progesterone agents. The 3-keto group on the hormone is not an ideal modification site because it interacts with a highly conserved region in the receptor protein (Andre & Pusztai (2006) Nat. Clin. Pract. Oncol. 3, 621-632., Madauss et al. (2004) J. Med. Chem. 47, 3381-3387., herein incorporated by reference in their entireties). Therefore, modification or isomerism at this site may compromise the binding affinity of the modified steroid (So et al. (2000) J. Chem. Inf. Comput. Sci. 40, 762-772., Bursi & Groen (2000) Eur. J. Med. Chem. 35, 787-796., Williams & Sigler (1998) Nature 393, 392-396., herein incorporated by reference in their entireties). A number of alternatives to this site are available and position 17 of the D ring was chosen.

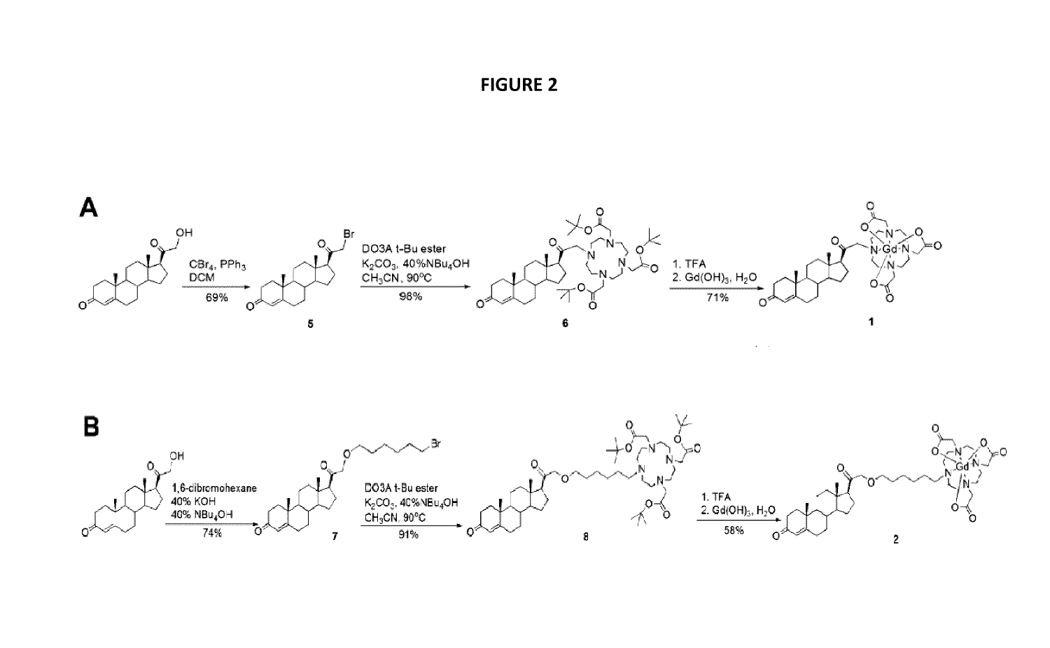

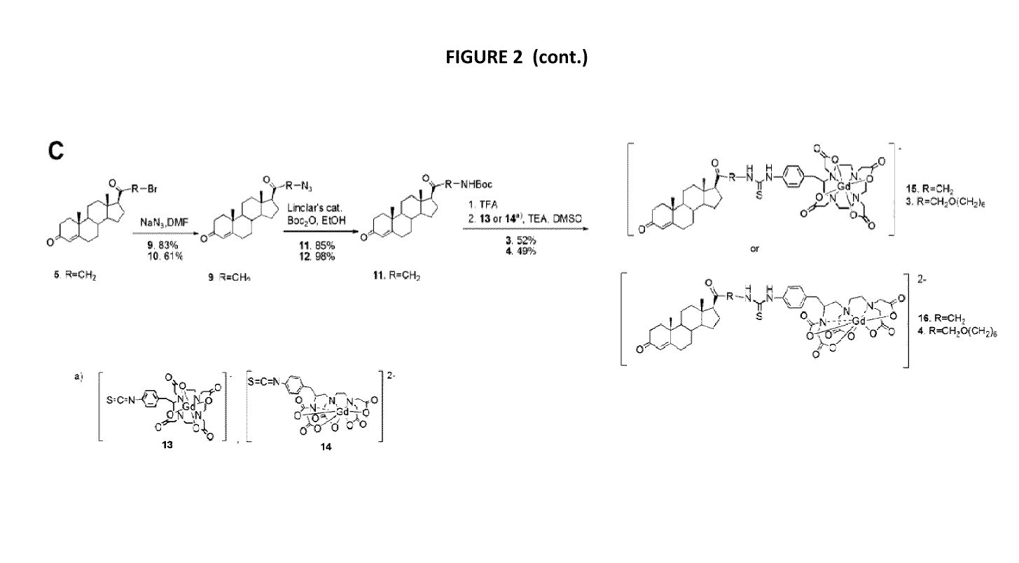

A series of Gd(III) complexes was synthesized to examine the effect of charge on lipophilicity and cell permeability of the conjugate (neutral, −1, and −2). Further, to determine the effect distance between the chelate and steroid may have on receptor binding affinity, spacers with varying lengths (zero, three, and six methylene carbons) were inserted between progesterone and the Gd(III) chelate. The neutral series of conjugates with zero- and six-carbon spacers (1, 2) was synthesized from 21-hydroxyprogesterone, as shown in FIGS. 2A and 2B. The synthesis of 1 began with bromination of the 21-hydroxyl group using carbon tetrabromide and triphenylphosphine. The attachment of 5 with K₂CO₃ (NBu₄OH as a catalyst) afforded the t-butyl-protected ligand in high yield (93%). After deprotection of the t-butyl group by trifluoroacetic acid, the ligand was heated with Gd(III) chloride at 60° C. to produce compound 1 in 72% yield. The synthesis of 2 started with alkylation of the 21-hydroxyl group by 1,6-dibromohexane. This biphasic alkylation reaction produced bromine-tethered progesterone (7) in 74% yield. A coupling reaction with DO3A followed by insertion of the lanthanide was performed by the same methods described in the synthesis of 2, with 58% yield.

The synthesis of the charged series of conjugates (3, 4) begins with the bromine intermediates (5, 7) from the neutral series (FIG. 2C). In the first step, the bromine group was substituted with an azide. After reducing the azide with Lindlar's catalyst, a coupling reaction of the free amine with an isothiocyanate-Gd(III) chelate was attempted. However, when the pendant amine is neutral or in basic form, the product is unstable, producing a number of uncharacterized byproducts. To circumvent this problem, the azide group was reduced and protected with a Boc group in one pot to give 13 and 14. After deprotection of the Boc group using trifluoroacetic acid, the TFA salt of amines was coupled with the charged Gd(III) chelates (15 and 16) to produce 3 and 4, respectively. Chelates possessing a −1 or −2 charge with no spacer (17 and 18) decomposed during preparative high-performance liquid chromatography purification, and the structure of these molecules was not determined. The charged Gd(III) chelates (15 and 16) were synthesized from commercially available ligands by previously published methods.

### Example 3

### Relaxivity and Octanol-Water Partition Coefficients

The relaxivity and octanol-water partition coefficients of 1-4 are presented in Table 1 and show that charged complexes have higher relaxivities. This tendency is due to aggregation caused by the amphiphilic nature of the compounds. The evidence supporting complex aggregation is the high relaxivity of 3 (19.1 mM⁻¹ s⁻¹) measured in the range of 0.125-2 mM. However, when the solution was diluted approximately 100-fold (concentration range from 0.001 to 0.1 mM), the relaxivity decreased to 5.9 mM⁻¹ s⁻¹.

The octanol-water partition coefficient (P) of each compound was measured to determine lipophilicity. The P value is often expressed in logarithmic form (logP), because the values usually range over many orders of magnitude. The observed logP values of the progesterone conjugates indicate that aggregation is occurring. Compound 3 has approximately the same logP value as 1; however, the relaxivities of these compounds are very different (19.1 and 3.77 mM⁻¹ s⁻¹, respectively). The results indicate that the overall lipophilicity of the two molecules is similar and that the charged species seem to be sufficiently amphiphilic to aggregate in solution. In addition, cellular uptake studies show that unlike commercially available Gd(III) contrast agents, such as PROHANCE, these new steroid conjugates are membrane permeable. Compared to PROHANCE (logP=−2) (Alvarez et al. (1997) J. Pharm. Sci. 86, 1187-1189., herein incorporated by reference in their entireties), all conjugates have greater logP values, demonstrating that lipophilicity of the molecules contributes to membrane permeability.

TABLE 1
Relaxivity Data and Octonol-Water Partition Coefficients (LogP)

| | | Relaxivity |
|---|---|---|
| Compound | LogP | (mM⁻¹s⁻¹)^{a} |
| Progesterone | 3.87^{b} | — |
| 1 | −0.292 | 3.77/4.76^{c} |
| 2 | 0.262 | 4.73c |
| 3 | −0.377 | 19.1/5.9^{d} |
| 4 | −0.959 | 6.5 |
| Prohance | −2.0 | 3.7* |

^{a}Data were measured at 60 MHz, 37° C.
^{b}data were taken from Alvarez et al.
^{c}Data were measured at 4.7 T, 21° C.
^{d}Data were measured at low concentration from 0.01 to 0.1 nM
*Data were taken from Caravan et al.

### Example 4

### Binding of Steroid-Gd(III) Conjugates Bind to PRA

Modification of a steroid with a Gd(III) chelate may interfere with the interaction of the steroid with its receptor. Competitive binding experiments were performed using increasing doses of the contrast agents and fluorescently labeled progesterone as the competitor, and the IC50 of each compound for PR was determined. The results are shown in Table 2. Relative binding of each progesterone contrast agent can be compared to progesterone. Compound 1 had the highest relative binding affinity for the receptor of all the compounds and differed only slightly from compound 3. Compound 2 had a relative affinity for the receptor that was about 100-fold lower than progesterone alone, but this was still in the high nanomolar range.

TABLE 2
Receptor Binding Affinity Results

| Compound | IC₅₀ (M) |
|---|---|
| Progesterone | 1.6 × 10⁻⁹ |
| 1 | 9.6 × 10⁻⁸ |
| 2 | 4.6 × 10⁻⁷ |
| 3 | 8.6 × 10⁻⁷ |
| RU-486* | 2.2 × 10⁻⁸ |
| RU-486-Gd* | 1.9 × 10⁻⁶ |

*Data were taken from Lee et al.

### Example 5

### Steroid-Based Contrast Agents Demonstrate Accumulated Gd(III) Inside Cells

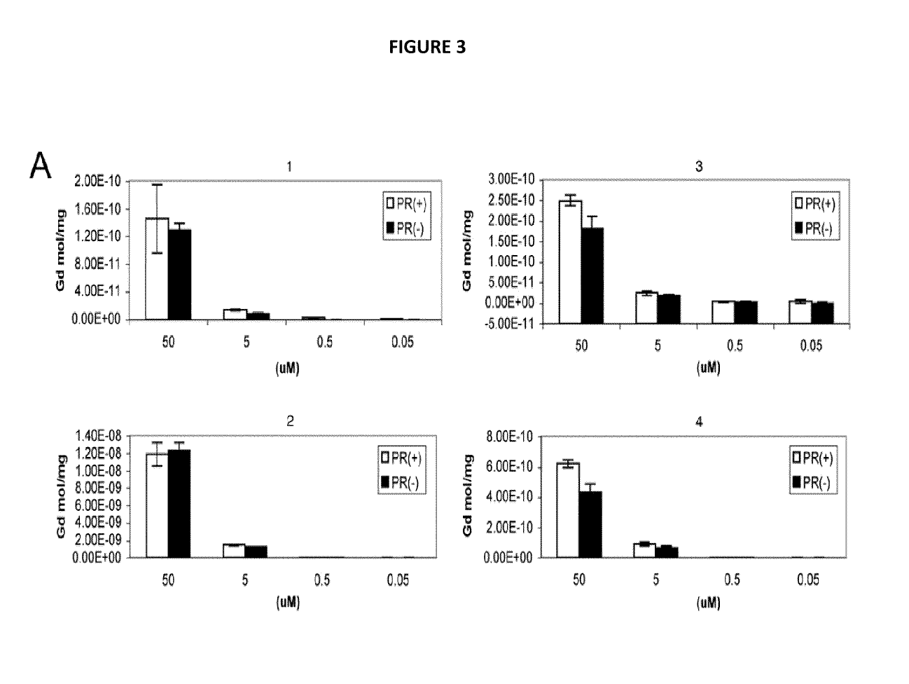

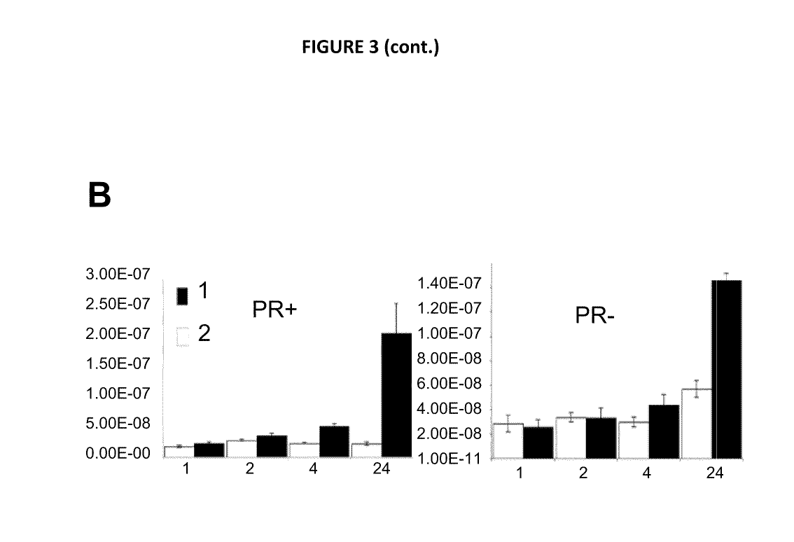

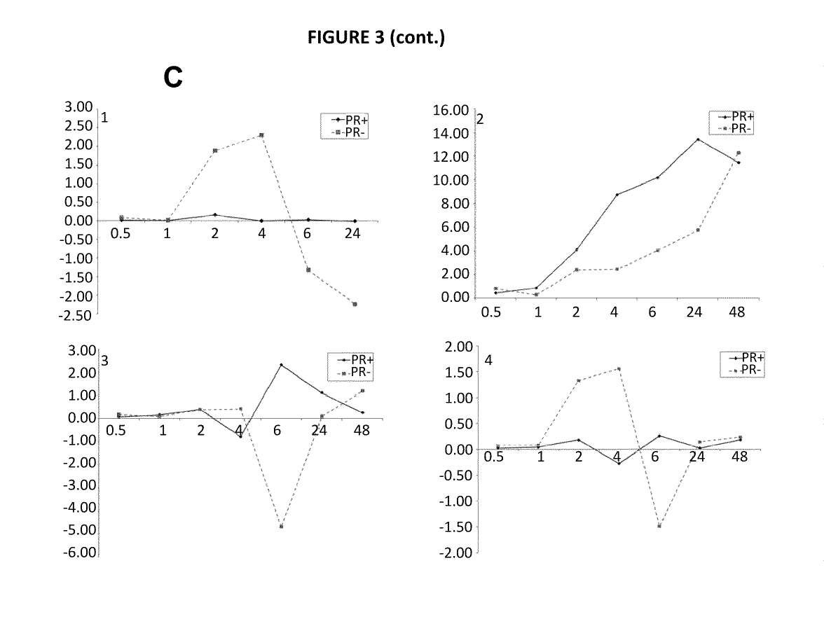

Cellular uptake experiments were performed by incubating the contrast agents with progesterone receptor positive (T47D) and progesterone receptor-negative (MDA-MB-231) mammary epithelial cells. In dose-response experiments cells were incubated with 50, 5, 0.5, or 0.05 mM agents for 24 hr with T47D and MDA-MB-231 cells. The contrast agents were absorbed in a dose-dependent manner and demonstrated maximal accumulation at 50 mM (FIG. 3A). Cells were incubated with only 1 or 2 at different time points including 1, 2, 4, and 24 hr. After incubation, the cells were lysed and subjected to inductively coupled plasma mass spectrometry (ICP-MS) analysis to quantify the number of Gd(III) ions present in each cell based on milligrams of protein (FIG. 3B). Compound 2 showed the highest uptake into the PR-expressing T47D cell line, and in cells lacking PR, the MDA-MB-231 cell line. Compounds 3 and 4 had much lower uptake, demonstrating that the negative charge of the contrast agent reduces cellular permeability. Similarly, compound 2 was significantly more absorbed than 1, demonstrating the importance of the hydrophobic carbon linker. To determine the cellular distribution of 1 and 2, synchrotron radiation X-ray fluorescence (SRXRF) analysis was performed. SR-XRF spectroscopy uses high-energy X-rays to produce a map of each element's concentration with submicrometer resolution, whereas conventional XRF analysis provides the elemental composition of materials. An advantage of SR-XRF over standard fluorescence microscopy is that images are obtained without altering the agent by attachment of an organic fluorophore. Compounds 1 and 2 (50 mM) were incubated with progesterone receptor-positive cells (T47D) and -negative cells (MDA-MB-231) for 24 hr prior to scanning. The samples were raster scanned with 2.0 μm×2.0 μm step size. The images show accumulation of each compound within cells and confirmed cellular uptake of 2.

### Example 6

### Steroid-Based Contrast Agents Efflux More Slowly from Receptor-Expressing Cancer Cells

One aspect of molecular specificity that a steroid-based contrast agent may provide is retention in cells expressing the progesterone receptor. Because progesterone interacts with its receptor and is active in the nucleus as a transcription factor long after initial absorption, experiments were designed to determine whether this would result in a slower leaching of the contrast agents from progesterone receptor-expressing T47D cells as compared to the progesterone receptor-negative MDA-MB-231 cells. Cells were incubated with 50 mM contrast agents for an initial 24 hr absorption period. The media containing the contrast agents were then removed, followed by three PBS washes, and the cells were then allowed to leach the intracellular portion of the contrast agent into serum-free media for 1, 2, 4, 6, 24, or 48 hr. The amount of the contrast agents was then determined by quantifying the amount of compound inside the cell using ICP-MS and then divided by the amount of gadolinium in the leached media minus the background (FIG. 3C). Although 1 did not appear to leach from the MDA-MB-231 cells more quickly, compound 2 showed much higher retention in the T47D cell line as compared to MDA-MB-231. Therefore, one way that these compounds specifically mark progesterone receptor-positive cells is by residing in the cell longer, due to interaction with PR.

### Example 7

### Steroid Conjugates Function to Transcriptionally Upregulate PRE-Luciferase

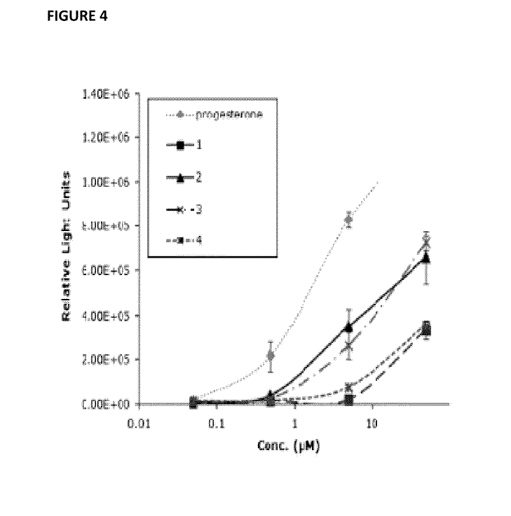

Progesterone receptors bind to a region of DNA referred to as the progesterone response element (PRE). This DNA element (repeated three times) was ligated to DNA encoding the luciferase gene and used to (1) monitor the cell permeability of the compounds, (2) verify the ability to interact with the progesterone receptor dimer, and (3) evaluate function in a transcription complex (FIG. 4). For the progesterone derivatives, induction of luciferase demonstrated that the compound was functional. Each compound showed the ability to alter transcription of the PRE, indicating that it entered the cell and bound to the full-length progesterone receptor. Agent 2 proved the most effective transcriptional agent and differed marginally from compound 3 in its ability to bind to the receptor. This demonstrates that a neutral charge on the agent is beneficial for activity and permeability. A hydrophobic linker between the progesterone molecule and the Gd(III) chelate appeared to improve cell permeability and may increase transcription, because the compounds are inside the cell interacting with the progesterone receptor for a longer period of time. These data confirm that although compound 2 does not bind as readily to the PR as compound 1, the improved cell permeability makes the agent enter the cells more rapidly, at lower doses, and provides higher transcriptional activity.

### Example 8

### Steroid-Based Contrast Agents Enhance T1 Relaxivity In Vitro

To determine the effect of 1 and 2 on T₁ after receptor binding, T₁-weighted images and measured spin-lattice relaxation times of incubated cells were obtained (FIG. 5A). Compound 1 was chosen because of its high binding affinity and compound 2 because of efficient cellular uptake. Progesterone receptor-positive cells (T47D, MDA-MB-231 transfected with PRA) and -negative cells (MDA-MB-231) were incubated with 50, 150, and 500 mM 1 and 2 for 24 hr prior to scanning. T₁-weighted images and relaxation times show that compound 2 enhanced MR contrast significantly more than compound 1 in any given cell type. All cells that were treated with compound 2 appeared much brighter than the cells that were treated with compound 1 or control media. For example, cells exposed to 150 mM 2 reduced T₁ more than 60% compared to controls. There are no significant changes in T₁ at 500 mM 2, demonstrating that the cells were saturated with compound 2 at 150 mM and no further uptake occurred.

### Example 9

### Steroid Hormones Modified with Gd(III) Chelates

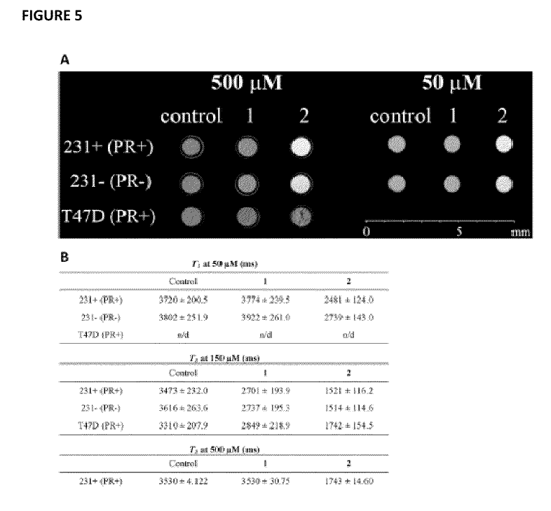

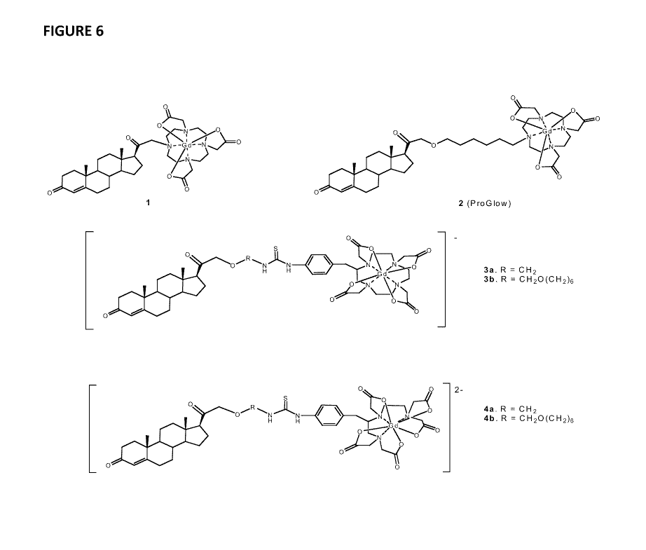

During experiments performed during development of embodiments of the present invention, first and second generation steroid-modified contrast agents, mifepristone-Gd(III) (RU486-Gd(III)) and progesterone-Gd(III) were developed. Progesterone can be monitored biologically, and has a higher binding affinity for the receptor and better activation of target genes than RU486-Gd(III). The progesterone contrast agents were synthesized by modifying 21-hydroxyprogesterone (SEE FIG. 6). The first progesterone contrast agent (SEE FIG. 6 (1)) synthesized has no linker between the Gd(III) chelate and progesterone. A second agent possesses a 6-carbon linker between the Gd(III) chelate and the progesterone molecule (SEE FIG. 6 (2)). Agents were prepared with a Gd(III) chelate that contains either a −1 or −2 charge (SEE FIG. 6 (3a, 3b, 4a, 4b)). This series of demonstrated higher water solubility while still traversing the phospholipid bilayer. Compound 2 demonstrated the best overall activity and image enhancement.

### Example 10

### Steroid Conjugates Bind to the Ligand Binding Domain of Progesterone Receptor A

Competitive binding experiments were carried out using fluorescently labeled progesterone as a competitor for each synthesized compound at increasing doses. Experiments were performed to determine the inhibitory concentration at 50% (IC₅₀) of each synthesized agent for the ligand-binding domain of the progesterone receptor (PR). The results for the compounds are shown in Table 3.

TABLE 3
Steroid contrast agents bind to the progesterone receptor.

| Compound | IC₅₀ (M) |
|---|---|
| Progesterone | 1.6 × 10⁻⁹ |
| RU486 | 2.2 × 10⁻⁸ |
| RU486-Gd(III) | 1.9 × 10⁻⁶ |
| 1 | 9.6 × 10⁻⁸ |
| 2 | 4.6 × 10⁻⁷ |
| 3a | 7.4 × 10⁻⁸ |
| 3b | 8.6 × 10⁻⁷ |

Values represent the inhibitory concentration at 50% (IC₅₀) for PR-Gd(III) contrast agents bound to the purified ligand-binding domain of progesterone receptor A.

Relative binding of each progesterone contrast agent was compared to progesterone, whereas RU486-Gd(III) was compared to RU486. The contrast agent with the highest affinity of those tested is compound 1 followed by compound 3a. All of the progesterone agents demonstrated at least a 10 fold higher binding affinity as compared to the RU486 agents.

### Example 11

### Cell Permeability of Steroid-Based Contrast Agents

Experiments were conducted during developments of embodiments of the present invention to develop cell permeable MRI contrast agents and to test the cell permeability of such agents. To test the cell-permeability of the synthesized compounds, cellular uptake experiments were performed by incubating the contrast agents with progesterone receptor positive (T47D) mammary epithelial cells. Dose response experiments were performed to identify the concentration of contrast agents needed for maximal cellular accumulation. The contrast agents were absorbed in a dose dependent manner and demonstrated maximal accumulation at 50 μM (not shown).

Cells were incubated with compound 1 compound 2 at various time points including 1, 2, 4, and 24 hours. After incubation, the cells were lysed, and subjected to ICP-MS analysis to quantify the amount of Gd(III) present in each cell based on mg of protein. Significantly more Compound 2 was absorbed into cells than Compound 1. This finding may demonstrate the importance of the hydrophobic carbon linker, although the present invention is not limited to any particular mechanism of action and an understanding of the mechanism of action is not necessary to practice the present invention.

### Example 12

### Steroid-Based Contrast Agents are Specifically Retained in Progesterone Receptor Cells

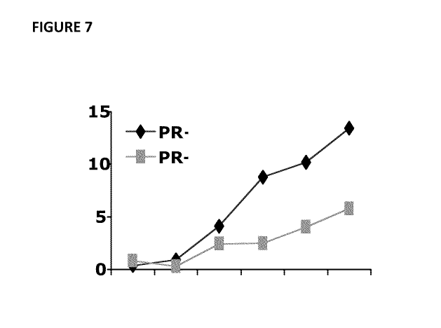

Progesterone mediated cell retention may provide specificity for imaging progesterone receptor positive mammary cancers. Experiments were performed during development of embodiments of the present invention to determine if progesterone binding resulted in slower leaching of the contrast agents from progesterone receptor expressing T47D cells as compared to the progesterone receptor negative MDA-MB-231 cells. Cells were incubated with 50 μM of compound 1 and compound 2 for an initial 24-hour absorption period. The media containing the contrast agents was then removed, followed by three PBS washes and then the cells were allowed to leach the intracellular portion of the contrast agent into serum free media for either 0.5, 1, 2, 4, 6, or 24 hours. The percent retained of the contrast agents was then determined by quantifying the amount of compound inside the cell using ICP-MS divided by the amount of Gd(III) in the leached media minus the background (SEE FIG. 7). Although compound 1 did not appear to leach from the MDA-MB-231 cells more quickly, compound 2 showed much higher retention in the T47D cell line as compared to MDA-MB-231. Compound 2 and other compound of the present invention specifically mark progesterone receptor positive cancers by residing in the receptor positive cells due to interaction with PR

### Example 13

### Steroid Conjugates Function to Transcriptionally Upregulate PRE-Luciferase

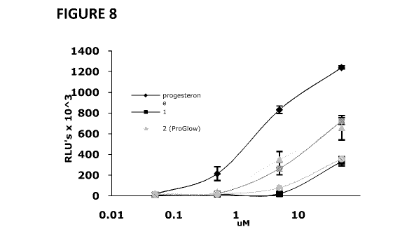

Progesterone receptors, when bound with progesterone, bind to a region of the DNA commonly referred to as the progesterone response element (PRE). A triple repeat of the PRE was ligated to DNA encoding the luciferase gene and was used to monitor cell permeability of the compounds as well as the ability of compounds to interact with a progesterone receptor and function as a transcriptional complex (SEE FIG. 8). The induction of luciferase indicated that the compound was functional. Each agent showed the ability to increase transcription of the PRE indicating that it entered the cell and bound to the full-length receptor. Compound 2 proved the most effective transcriptional agent and only differed marginally from compound 3a in its ability to bind to the receptor, thus indicating that a neutral charge on the contrast agent is most beneficial for activity and permeability. The transcription of PRE in response to these novel contrast agents demonstrates: 1) the agents cross the cell membrane, 2) the agents bind to the full-length endogenous receptor and initiate dimerization, and 3) the agents function as initiators of gene transcription which will likely provide for longer cell retention and demonstrates that the biological target has been activated.

### Example 14

### Steroid-Based Contrast Agents Enhance Relaxivity in Mammary Cells

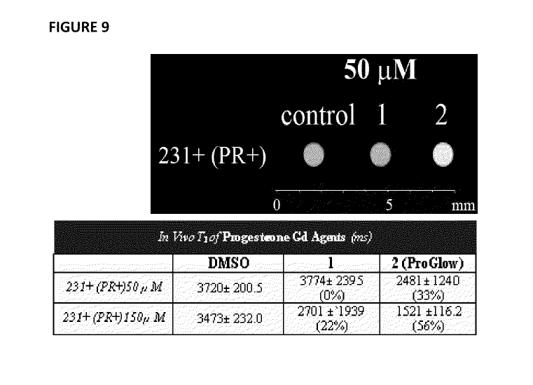

T₁-weighted images and measured spin-lattice relaxation times were obtained of cells incubated with compound 1 and compound 2 (SEE FIG. 9). Compound has demonstrated a high binding affinity and compound 2 has demonstrated efficient cellular uptake. Progesterone receptor positive cells (MDA-MB-231 transfected with PR-A) were incubated with 50, 150, and 500 uM of compound 1 and compound 2 for 24 hours prior to scan. The images are of cell pellets that were grown in monolayer culture, treated with contrast agent, washed with PBS, and then compacted into a capillary tube for MRI. The T₁ values were weighted. All MR data was collected at ambient temperature in a General Electric/Bruker Omega 400WB 9.4 T magnet (83 mm bore size) fitted with Accustar shielded gradient coils. Spin lattice relaxation times (T₁) were measured using an inversion recovery pulse sequence. Images were acquired using a T₁ weighted spin-echo pulse sequence with repetition time (T_{R}) of 100˜600 ms and an echo time (T_{E}) of 10˜10.2 ms. T₁-weighted images and relaxation times show that compound 2 enhanced MR contrast significantly more than compound 1. All cells that were treated with compound 2 appeared brighter than the cells that were treated with compound 1 or control media. The synthesized progesterone compounds significantly increase relaxivity of cells in vitro.

### Example 15

### Steroid-Based Contrast Agents Accumulate and Specifically Enhance Relaxivity in the Uterus and Ovaries

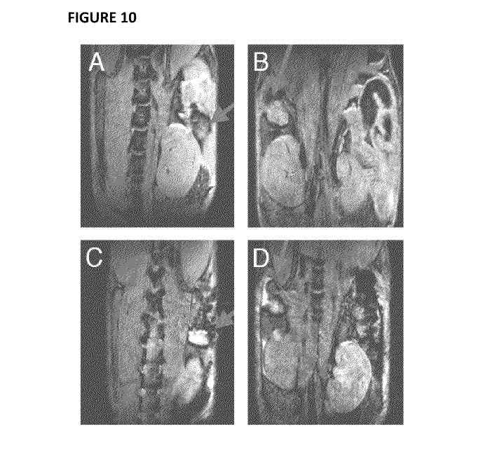

Experiments were performed during development of embodiments of the present invention to examine the accumulation of compound 2 in the progesterone receptor rich ovarian tissues of a female mouse in vivo. Compound 2 was suspended in DMSO and injected in the intraperitoneal cavity at a concentration of 0.15 mmol/kg weight of the mouse. The mouse was imaged prior to contrast agent to create the untreated, negative control (SEE FIGS. 10A and B). The ovaries were then identified using gradient echo imaging TR/TE (250/7 ms), array size 256², and slice thickness 0.5 mm, NEX 2. The post injection images were acquired after 30 minutes (SEE FIGS. 10C and D). The ovary and uterus specifically retain compound 2 after injection. In addition, compound 2 generates a significant increase in relaxivity producing a vibrant image of a progesterone rich organ in the mouse.

### Example 16

### Steroid-Based Contrast Agents are Retained in Tissues that Express Progesterone Receptors In Vivo

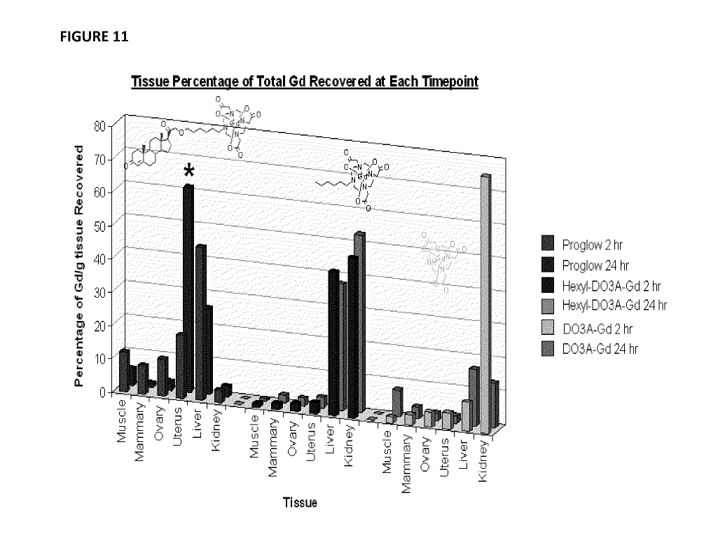

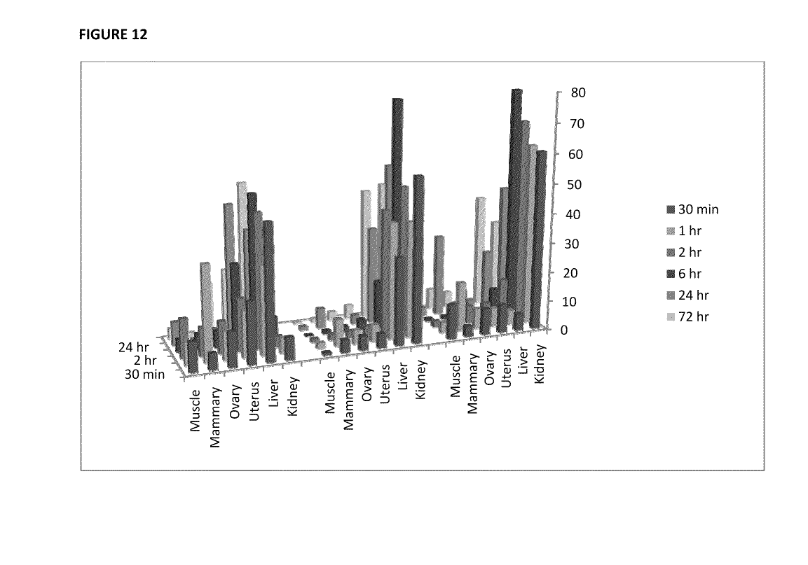

A biodistribution study performed during development of embodiments of the present invention to determine whether steroid-modified contrast agents are specifically retained within cell types that express the progesterone receptor. Compound 2 was suspended in DMSO and injected into mice and the organs were harvested at 30 minute and 1, 2, 6, 24, and 72 hour timepoints. The harvested organs were dissolved by heating in concentrated nitric acid, and the Gd(III) content was determined by ICP-MS. The data was compared to biodistribution of two control compounds, hexyl-DO3A-Gd(III) (Compound 2 without the ligand moiety) and DO3A-Gd(III) (the contrast moiety of c). Data was acquired at the 30 minute, 1 hour, 2 hour, 6 hour, and 24 hour time points (SEE FIGS. 11 and 12). Gd(III) is shown as a percentage of the total Gd(III) per gram of tissue recovered from all the organs at each timepoint. The control compounds do not target any tissues and mainly end up in the liver and kidney, whereas compound 2 is retained in the uterus, which expresses PR.

Experiments were performed during development of embodiments of the present invention have demonstrated that steroid-based Gd(III) contrast agents can be synthesized, bind to their respective receptors, cross mammalian cellular membranes, drive transcription, and provide a magnetic resonance signal. The progesterone contrast agents are more specific and more cell permeable than the RU486 compounds.

All publications and patents mentioned in the present application are herein incorporated by reference. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A composition comprising: a) a ligand moiety, b) a contrast moiety, and c) a linkage region, wherein said linkage region covalently links said ligand moiety to said contrast moiety.

2. The composition of claim 1, wherein said ligand moiety comprises a hormone.

3. The composition of claim 2, wherein said hormone comprises progesterone.

4. The composition of claim 1, wherein said contrast moiety comprises a metal-ion chelator.

5. The composition of claim 4, wherein said metal-ion chelator comprises DO3A.

6. The composition of claim 4, wherein said metal-ion chelator coordinates a paramagnetic metal ion.

7. The composition of claim 6, wherein said paramagnetic metal ion is selected from the group comprising Gd(III), Fe(III), Mn(II), Y(III), Cr(III), Eu(III), and Dy(III).

8. The composition of claim 7, wherein said paramagnetic metal ion comprises Gd(III).

9. The composition of claim 1, wherein said linker region comprises one or more methylene carbons.

10. The composition of claim 9, wherein said linker region comprises 3 or 6 methylene carbons.

11. The composition of claim 1, wherein said linker region comprises a covalent bond between said contrast moiety and said ligand moiety.

12. A composition comprising: a) a hormone, b) metal-ion chelator, and c) a linkage region, wherein the linkage region covalently links said hormone to said metal-ion chelator.

13. The composition of claim 12, wherein said hormone comprises progesterone.

14. The composition of claim 13, wherein said metal-ion chelator coordinates a paramagnetic metal ion.

15. The composition of claim 14, wherein said paramagnetic metal ion comprises Gd(III).

16. A method comprising:
a) administering a composition of claim 15 to a cell or tissue; and
b) producing a magnetic resonance image of said cell or tissue.

17. The method of claim 16, wherein said cell or tissue expresses progesterone receptor.

18. The method of claim 17, wherein said progesterone binds to said progesterone receptor.

19. The method of claim 18, wherein binding of said progesterone to said progesterone receptor results in localization of said composition.

20. The method of claim 16, wherein said cell or tissue does not express progesterone receptor.
